(19) United States
(12) Patent Application Publication
(10) Pub. No.: US 2024/0050078 A1
(43) Pub. Date: Feb. 15, 2024

(54) CHOLANGIOSCOPE SYSTEM GUIDE SHEATH AND ANCHOR WIRE

(71) Applicant: GYRUS ACMI, INC. D/B/A OLYMPUS SURGICAL TECHNOLOGIES AMERICA, Westborough, MA (US)

(72) Inventors: Nikhil M. Murdeshwar, Maple Grove, MN (US); Thomas J. Holman, Princeton, MN (US)

(21) Appl. No.: 18/257,809

(22) PCT Filed: Nov. 9, 2021
(86) PCT No.: PCT/US2021/058561

§ 371 Date: Jun. 15, 2023

Related U.S. Application Data
(60) Provisional application No. 63/199,316, filed on Dec. 18, 2020.
(60) Provisional application No. 63/213,849, filed on Jun. 23, 2021.

(51) Int. Cl. A61B 10/06; A61B 1/00; A61B 1/018; A61B 1/273; A61B 17/29

(52) U.S. Cl. CPC A61B 10/06; A61B 1/00135; A61B 1/018; A61B 1/2736; A61B 17/29; A61B 1/00148

(57) ABSTRACT

A direct peroral cholangioscopy method comprises inserting an endoscope into a guide sheath, inserting the guide sheath and endoscope into a duodenum, extending a tissue retrieval device through the endoscope into the duodenum, extending an anchor wire into the duodenum, collecting biological matter with the tissue retrieval device, and withdrawing the cholangioscope and tissue retrieval device from the duodenum along the anchor wire. A cholangioscopy system comprises a steerable guide sheath and a cholangioscope comprising a shaft extending to a distal end face for displacement within the steerable guide sheath, a tool lumen extending along the shaft and exiting at the distal end face, an anchor lumen entering the shaft between a proximal end portion and the distal end face and exiting at the distal end face, and an exchange lumen extending from the tool lumen to an exterior of the shaft at the distal end face.

### CROSS-REFERENCE TO RELATED APPLICATIONS

This application claims the benefit of priority to U.S. Provisional Patent Application Ser. No. 63/199,316, filed Dec. 18, 2020; and U.S. Provisional Patent Application Ser. No. 63/213,849, filed Jun. 23, 2021, the contents of which are hereby incorporated by reference in their entireties.

### TECHNICAL FIELD

The present disclosure relates generally to medical devices comprising elongate bodies configured to be inserted into incisions or openings in anatomy of a patient to provide diagnostic or treatment operations.

More specifically, the present disclosure relates to medical devices, such as endoscopes, that can be inserted into anatomy of a patient, such as with the aid of another device, to facilitate performance of a biological matter removal process, such as by cutting sample tissue with a forceps for later analysis.

### BACKGROUND

Endoscopes can be used for one or more of 1) providing passage of other devices, e.g., therapeutic devices or tissue collection devices, toward various anatomical portions, and 2) imaging of such anatomical portions. Such anatomical portions can include gastrointestinal tract (e.g., esophagus, stomach, duodenum, pancreaticobiliary duct, intestines, colon, and the like), renal area (e.g., kidney(s), ureter, bladder, urethra) and other internal organs (e.g., reproductive systems, sinus cavities, submucosal regions, respiratory tract), and the like.

Conventional endoscopes can be involved in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, and providing suction passageways for collecting fluids (e.g., saline or other preparations) and the like.

In conventional endoscopy, the distal portion of the endoscope can be configured for supporting and orienting a therapeutic device, such as with the use of an elevator. In some systems, two endoscopes can be configured to work together with a first endoscope guiding a second endoscope inserted therein with the aid of the elevator. Such systems can be helpful in guiding endoscopes to anatomic locations within the body that are difficult to reach. For example, some anatomic locations can only be accessed with an endoscope after insertion through a circuitous path.

One example of an endoscopic procedure is called an Endoscopic Retrograde Cholangio-Pancreatography, hereinafter “ERCP” procedures. In an ERCP procedure, an auxiliary scope (also referred to as daughter scope, or cholangioscope) can be attached and advanced through the working channel of a “main scope” (also referred to as mother scope or duodenoscope). Furthermore, a tissue retrieval device used to remove the sample matter is inserted through the auxiliary scope. As such, the duodenoscope, auxiliary scope and tissue retrieval device become progressively smaller, due to being sequentially inserted in progressively smaller lumens, and more difficult to maneuver and perform interventions and treatments.

### SUMMARY

The present inventors have recognized that problems to be solved with conventional medical devices, and in particular endoscopes and duodenoscopes used to retrieve sample biological matter, include, among other things, 1) the difficulty in navigating endoscopes, and instruments inserted therein, to locations within anatomical regions deep within a patient, 2) the disadvantage associated with operating three instruments (e.g., duodenoscope, cholangioscope, tissue removal device) such as the need for multiple skilled instrument operators, 3) the increased time and associated cost of having to repeatedly remove and reinsert medical devices to obtain a sufficient quantity of sample material, and 4), the difficulty of incorporating features (e.g., steerability and tissue collection features) into small-diameter devices.

The present inventors have recognized that such problems can be particularly present in duodenoscopy procedures such as the aforementioned ERCP procedures. Recent attempts have been made to address the deficiencies of ERCP procedures. Recent developments have involved the use of endoscopes in direct peroral cholangioscopy procedures where an endoscope is advanced directly into the mouth of a patient to reach the common bile duct. See for example the discussion in Direct Peroral Cholangioscopy by Mansour A. Parsi, MD, in World Journal of Gastrointestinal Endoscopy published online Jan. 16, 2014. However, such procedures are difficult to perform due looping of the endoscope produced by navigating the endoscope through the pyloric sphincter of the stomach and the sphincter of the common bile duct (Sphincter of Oddi). This looping of the endoscope can result in inoperability of the endoscope due to binding, e.g., the endoscope is too tightly curved to allow for additional articulation.

The present disclosure can help provide solutions to these and other problems by providing systems, devices and methods relating to direct peroral cholangioscopy procedures, such as those including a guide sheath that can be placed over an endoscope to provide 1) direct secondary steerability of the endoscope in addition to the primary native steering capabilities of the endoscope and 2) a rigid structure against which the endoscope can be pushed using the primary native steering capabilities to provide for articulation of the endoscope. As such, when the endoscope is positioned in the common bile duct, the native steerability features of the endoscope remain operable to position a therapeutic device at target tissue sites (e.g., sites where tissue to be treated or retrieved for analysis resides).

As such, the present disclosure can help solve the problems referenced above and other problems by 1) increasing the ease of use of direct per oral cholangioscopy systems and ERCP systems (e.g., fewer operators, less required skill to navigate through the Sphincter of Oddi/ampulla of Vater), and 2) increasing treatment device size (e.g., increasing the volume of sample material collected with each insertion, reducing the number of times a tissue retrieval device needs to be inserted and reinserted into the anatomy), among other things, as is described herein.

In an example, a cholangioscopy system can comprise a guide sheath and a cholangioscope. The guide sheath can comprise a steerable lumen. The endoscope can comprise an elongate shaft extending between a proximal end portion and a distal end face, the elongate shaft configured for displacement along the steerable lumen, a working tool lumen extending along the elongate shaft and exiting at the distal end face, an anchor lumen entering the elongate shaft between the proximal end portion and the distal end portion and exiting the elongate shaft at the distal end face, and a non-axial lumen extending from the working tool lumen to an exterior of the elongate shaft at the distal end face.

In another example, a method of performing a direct peroral cholangioscopy procedure can comprise inserting an endoscope into a guide sheath, inserting the guide sheath and endoscope into the duodenum of a patient, extending a tissue retrieval device through the endoscope into the common bile duct of the patient, extending an anchor wire into the common bile duct, collecting biological matter with the tissue retrieval device, and withdrawing the cholangioscope and tissue retrieval device from the common bile duct along the anchor wire.

Furthermore, the present inventors have recognized that problems to be solved with conventional surgical procedures, such as duodenoscopy procedures in the gastrointestinal system, sometimes utilize a follow-up procedure to address issues from the original procedure or to provide additional treatment. Sometimes multiple follow-up procedures are performed, such as to provide ongoing treatment. As such, the complicated navigation and steering procedures described above are repeated a second or multiple times.

The present disclosure provides solutions to these and other problems by providing implantable devices that can facilitate follow-up procedures. In particular, the implantable devices can expedite follow-up procedures by providing quick access to anatomy, such as the duodenum and particularly the common bile duct. The reentry device can comprise a stent that can include cutting capabilities to allow the stent to be implanted in anatomic constrictions. The implantable devices can include reentry devices comprising tubes, tracks, rails, guidewires and the like that can provide a pathway through complex anatomical geometries to allow procedures to follow a previously plotted path. The reentry devices can allow for a treatment device, such as a stent, to be inserted into a reentry tube or slid over a reentry guidewire, thereby eliminating the need for complicated mother/daughter scopes and the like. The treatment devices can be implanted into the anatomy to provide one-time or ongoing treatment, such as pumping of fluid, eradicating of stones and the like.

In an example, a system for providing repeatable entry into an anatomic area of a patient can comprise a stent comprising an annular body and a reentry track extending through the stent that can comprise an elongate body comprising proximal and distal ends extending out of the annular body.

In another example, a method for implanting a treatment device in anatomy can comprise implanting a stent in an anatomic opening, positioning a reentry track extending from the stent into an anatomic passageway, sliding the treatment device along the reentry track to position the treatment device in the anatomic passageway and treating the anatomy with the treatment device.

### BRIEF DESCRIPTION OF DRAWINGS

FIG. 1A is a schematic diagram of a direct peroral cholangioscope system comprising a guide sheath, a cholangioscope and a tissue retrieval device in a nested configuration.

FIG. 1B is a schematic diagram of the direct peroral cholangioscope system of FIG. 1A in an exploded state showing internal lumens through the guide sheath and the cholangioscope.

FIG. 2 is a schematic diagram of the distal end portions of the guide sheath, the cholangioscope, and the tissue retrieval device of FIGS. 1A and 1B.

FIG. 3 is a schematic diagram of the cholangioscope of FIGS. 1A and 1B showing a schematic representation of an imaging and control system comprising a control unit connected to the cholangioscope.

FIG. 4 is schematic diagram of the imaging and control system of FIG. 3 connected to the cholangioscope.

FIG. 5A is an end view of a camera module including optical and functional components suitable for use with the cholangioscope of FIGS. 1A-4.

FIG. 5B is a cross-sectional view taken along the plane 5B-5B of FIG. 5A showing components of the camera module.

FIG. 6 is a schematic diagram illustrating the guide sheath and the cholangioscope of FIGS. 1A and 1B inserted perorally into a patient to reach the duodenum.

FIG. 7 is a schematic diagram illustrating the guide sheath and the cholangioscope of FIG. 6 positioned in the duodenum and the tissue retrieval device positioned in the common bile duct.

FIG. 8 is perspective view of another example of a cholangioscope configured for use with the guide sheath of the present disclosure.

FIG. 9 is an end view of the cholangioscope of FIG. 8 showing an anchor wire lumen and a non-axial lumen.

FIG. 10 is a schematic cross-sectional view taken along the plane 10-10 of the cholangioscope of FIG. 9 showing the non-axial lumen.

FIG. 11 is a schematic cross-sectional view taken along the plane 11-11 of the cholangioscope of FIG. 9 showing the anchor wire lumen.

FIG. 12A is schematic diagram of the cholangioscope of FIG. 8 inserted into a common bile duct with the anchor wire attached to tissue and the tissue retrieval device extended beyond the non-axial lumen.

FIG. 12B is a schematic diagram of the cholangioscope of FIG. 8 withdrawn from the common bile duct with the anchor wire attached to tissue and the tissue retrieval device bent to extend through the non-axial lumen.

FIG. 13 is a block diagram illustrating methods of retrieving tissue samples from within a common bile duct of a patient using a cholangioscope having an anchor wire lumen and a non-axial lumen.

FIG. 14 is a schematic view of a duodenum connected to a common bile duct via a duodenal papilla.

FIG. 15 is a schematic view of the duodenum of FIG. 14 with a stent of the present disclosure inserted into the duodenal papilla.

FIG. 16A is a schematic view of a collapsed stent having an inflation balloon inserted therein and electric leads extending therethrough.

FIG. 16B is a schematic view of the stent of FIG. 16A in an expanded state with the electric leads in situ.

FIG. 16C is a schematic view of the stent of FIG. 16B in an expanded state with the electric leads pulled away from the stent.

FIG. 17 is a schematic side view of a stent of the present application including electric leads extended into a cylindrical mesh stent body.

FIG. 18 is a schematic side view of a stent of the present application including mechanical cutting edges.

FIG. 19 is an end view of the stent of FIG. 18 showing anchoring barbs.

FIG. 20A is a schematic cross-sectional view of a magnetically-activated stent in a collapsed state.

FIG. 20B is a schematic cross-sectional view of the magnetically-activated stent of FIG. 20 is an expanded state.

FIG. 21 depicts an example of a magnetic applicator configured to expand and contract the magnetically-activated stent of FIGS. 20A and 20B.

FIG. 22 is schematic side view of a stent of the present application including extendable elongate reentry devices.

FIG. 23 is an end view of the stent of FIG. 22 showing lumens through the stent and extendable reentry devices.

FIG. 24 is a schematic view of the stent of FIGS. 22 and 23 with the extendable elongate reentry devices in a deployed state.

FIG. 25 is a side view of a stent of the present disclosure having an elongate reentry device with a funnel entrance as deployed in a gallbladder duct.

FIG. 26 is a schematic side view of a stent comprising elongate reentry devices including distal stents.

FIG. 27 is a schematic side view of a duodenum and the stent of FIG. 26 deployed within a gallbladder duct and a pancreatic duct.

FIG. 28 is a schematic view of a duodenum having an implantable device of the present disclosure including an expandable and contractable pumping stent.

FIG. 29A is a schematic side view of a stent configured to process gall stones.

FIG. 29B is a schematic end view of the gall stone processing stent of FIG. 29A.

FIG. 30 is a schematic view of a duodenum having an implantable device of the present disclosure including a plurality of monorail devices.

FIG. 31 is a schematic view of a monorail comprising a corkscrew anchor.

FIG. 32A is a schematic view of a monorail comprising a first example of a deployable anchor in a collapsed state.

FIG. 32B is a schematic view of the monorail of FIG. 32A with the deployable anchor in an extended state.

FIG. 33A is a schematic view of a monorail comprising a second example of a deployable anchor in a collapsed state.

FIG. 33B is a schematic view of the monorail of FIG. 33A with the deployable anchor in an extended state.

FIG. 34 is a schematic illustration of a first example of an elongate deployable member comprising a perforation.

FIG. 35 is a schematic illustration of a first example of an elongate deployable member comprising a slit.

FIG. 36 is a schematic illustration of a first example of an elongate deployable member comprising a gap.

FIG. 37 is a schematic illustration of a first example of an elongate deployable member comprising c-shaped reinforcements.

FIG. 38 is a schematic illustration of a first example of an elongate deployable member comprising a magnetic closure element.

FIG. 39 is a schematic perspective view of an elongate deployable member having a treatment device being slid therethrough to open the elongate deployable member.

FIG. 40 is a block diagram illustrating methods of implanting a reentry device of the present application having treatment devices.

### DETAILED DESCRIPTION

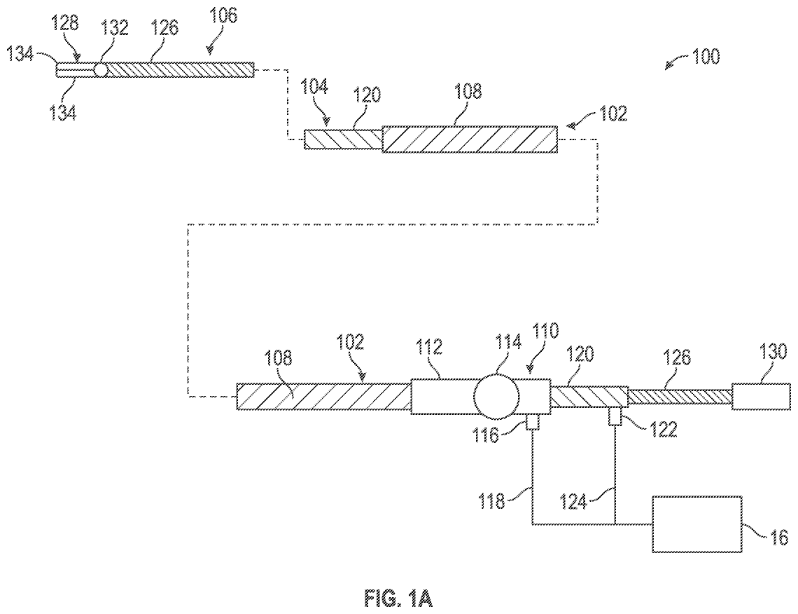

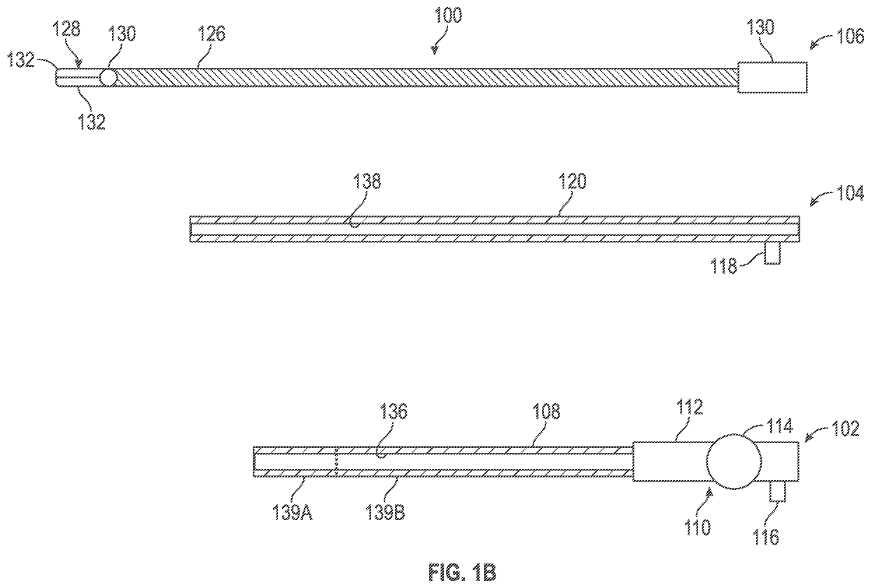

FIG. 1A is a schematic diagram of direct peroral cholangioscopy system 100 wherein guide sheath 103, cholangioscope 104 and tissue retrieval device 106 are in a nested configuration. FIG. 1B is a schematic diagram of direct peroral cholangioscopy system 100 wherein guide sheath 103, cholangioscope 104 and tissue retrieval device 106 are in an exploded configuration. FIGS. 1A and 1B are discussed concurrently. FIGS. 1A and 1B are not necessarily drawn to scale and may be exaggerated in certain aspects for illustrative purposes.

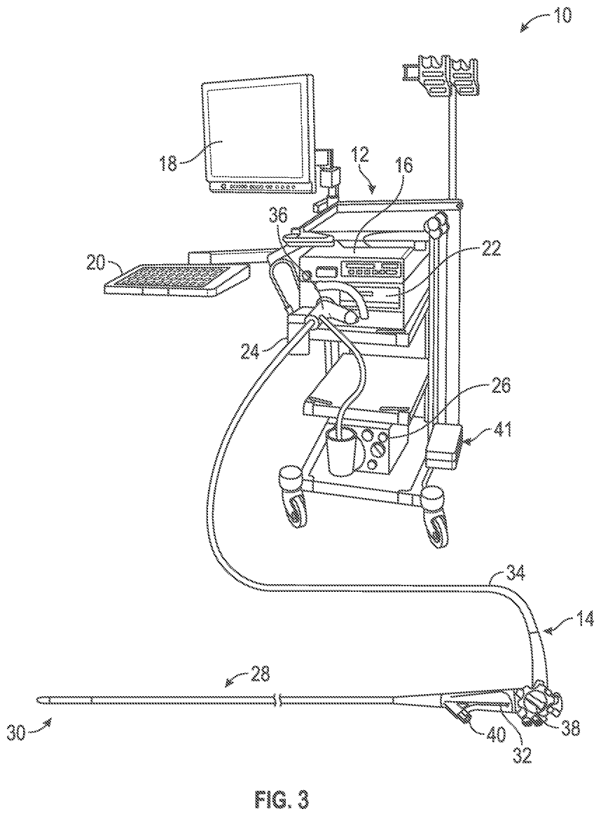

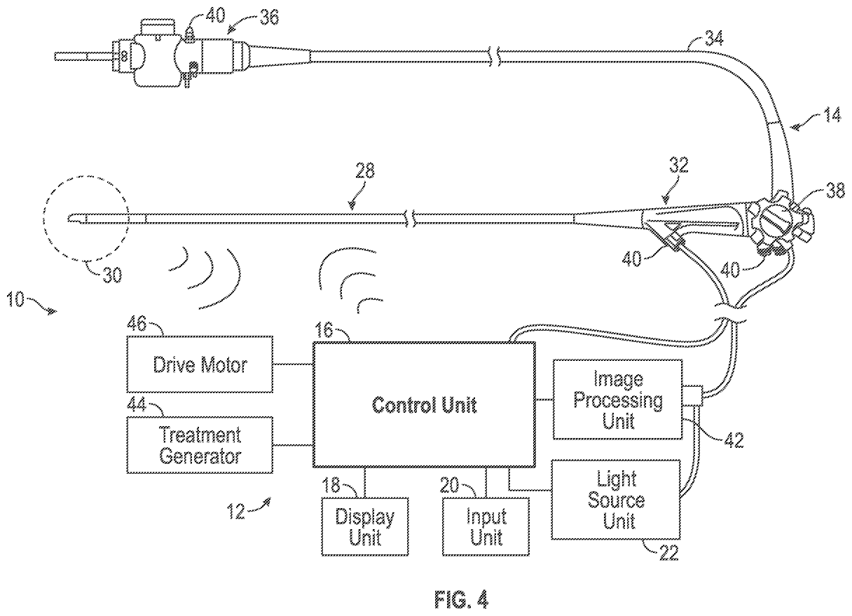

System 100 can comprise guide sheath 102, cholangioscope 104 and tissue retrieval device 106. Sheath 102 can comprise shaft 108 and control device 110, which can include grip 112, control knob 114 and coupler 116 that can connect to control unit 16 (FIG. 4) via cable 118. Cholangioscope 104, which is described in greater detail with reference to FIGS. 3-5B, can comprise elongate body 120 and coupler 122 that can connect to control unit 16 via cable 124. Tissue retrieval device 106 can comprise shaft 126, tissue separator 128 and control device 130. Tissue separator 128 can comprise hinge 132 and separators 134.

FIG. 1A shows cholangioscope 104 nested inside of sheath 102, and tissue retrieval device 106 nested inside cholangioscope 104. As such, as can be seen in FIG. 1B, sheath 102 can comprise lumen 136 and cholangioscope 104 can comprise lumen 138.

As is discussed in greater detail herein, direct peroral cholangioscopy system 100 can be configured to provide simplified navigation to a duodenum and common bile duct, and allow for large sample sizing, thereby reducing procedure complexity and the number of times that an instrument is inserted into the duodenum and common bile duct to retrieve an adequate amount of tissue upon which testing can be performed.

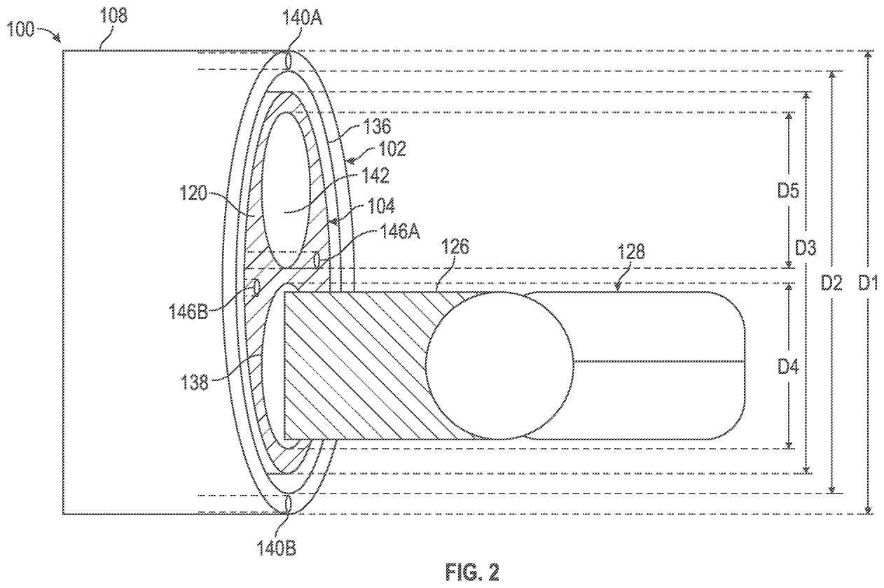

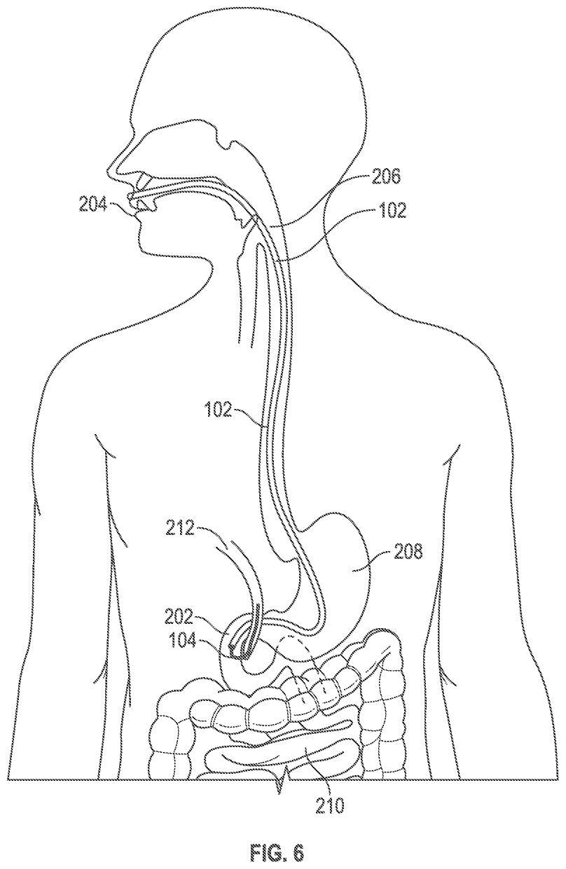

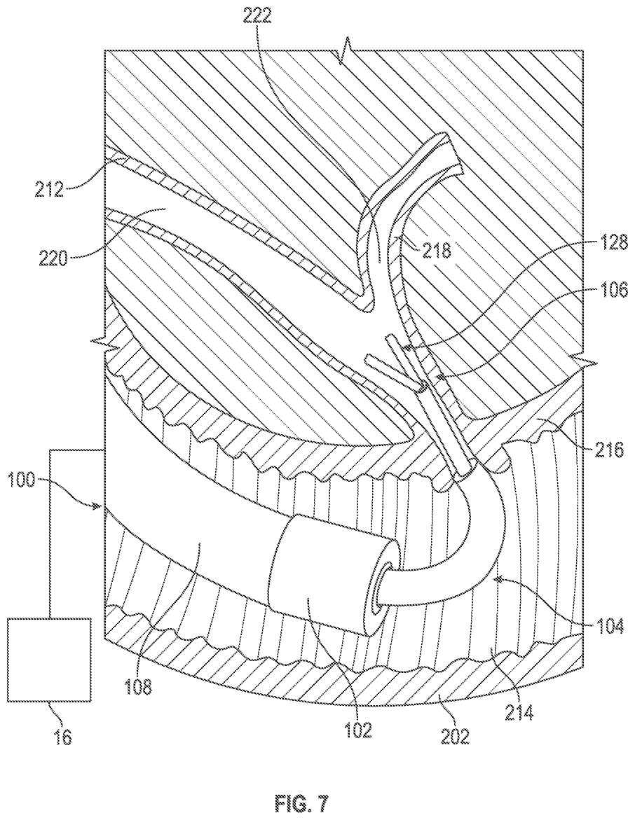

Shaft 108 of guide sheath 102 can include pull wires (140A, 140B of FIG. 2) that can be used to steer guide sheath 102. Control device 110 can be used to operate guide sheath 102, including the pull wires. For example, grip 112 can be grasped by an operator and control knob 114 can be rotated to pull on one or both of the pull wires to apply directionality to the shape of shaft 108. Guide sheath 102 can thus be used to influence the shape of cholangioscope 104 that can be positioned inside of shaft 108. Thus, shaft 108 can be made of suitable materials compliant enough to be directed by pull wires, but rigid enough to allow cholangioscope 104 to push off of shaft 108. In examples, shaft 108 can be configured in sections having different rigidities to, for example, concentrate the ability of shaft 108 to be steered in a particular section of guide sheath 102. For example, distal end portion 139A of shaft 108 (e.g., approximately the most-distal 10 ten to twenty percent) can be configured to be more flexible than the remaining proximal portion 139B so that the pull wires can pull the distal end of shaft 108 at a sharp angle (e.g., approximately thirty to ninety degrees), which can be useful in guiding cholangioscope 104 into common bile duct 212 (FIG. 7) from duodenum 202 (FIG. 7). Sectioning of the flexibility of shaft 108 can allow more rigid portions proximal of the distal flexible portion to provide stiffening to cholangioscope to allow cholangioscope to use native steering capabilities more effectively (e.g., without binding as described above). For example, proximal portion 139B can have a rigidity that is more rigid than cholangioscope 104 so that when cholangioscope 104 is steered, e.g., by tensioning of one or more pull wires 146A and 146B, cholangioscope 104 can be pushed into the desired direction by the pulling of pull wires 146A and 146B.

Cholangioscope 104 can be configured as a fully functional endoscope including steerability, guidance capability, imaging capability, fluid dispensing and retrieving capabilities, and functional (e.g., therapeutic and diagnostic) capabilities, as well as a passageway for other instruments. Functionality of cholangioscope 104 is described in detail with reference to endoscope 14 of FIGS. 3 and 4 below and, as such, is only shown schematically in FIGS. 1A and 1B.

Tissue retrieval device 106 can be configured as any suitable device configured to obtain tissue samples form within a patient. However, tissue retrieval device 106 can comprise a component or device for interacting with a patient, such as those configured to cut, slice, pull, saw, punch, twist or auger tissue, and the like. Specifically, tissue retrieval device 106 can comprise any device suitable for removing tissue from a patient, such as a blade, punch or an auger. Tissue retrieval device 106 can be configured to physically separate portions of tissue of a patient from other larger portions of tissue in the patient. In additional examples, tissue retrieval device 106 can be configured to simply collect biological matter from the patient that does not need physical separation, such as mucus or fluid. In the illustrated example, tissue retrieval device 106 can comprise forceps having separators 134 configured as sharpened or serrated jaws pivotably connected at hinge 132. Tissue retrieval device 106 can, however, be configured as a variety of devices capable of collecting biological matter, such as a punch, an auger, a blade, a saw and the like, as mentioned. The term “tissue retrieval device” is used throughout the present disclosure, however tissue retrieval device 106 can alternatively or additionally comprise a biological matter collection device, a biological matter retrieval device, a tissue collection device and tissue retrieval device.

Tissue retrieval device 106 can be configured to hold a volume of collected biological matter, e.g., tissue, such as between separators 134. As such, tissue retrieval device 106 can be configured to be withdrawn from cholangioscope 104 to obtain the collected biological matter, such as for diagnostic analysis. In other examples, tissue retrieval device 106 can include a lumen through which biological matter can be withdrawn without having to withdraw tissue retrieval device 106 from cholangioscope 104.

Guide sheath 102 can be configured as a simple tubular body with limited navigation capabilities that can be used to enhance native navigation capabilities of a cholangioscope, without increasing the size and complexity of the system components and the procedure, thus allowing the native navigation and imaging capabilities of cholangioscope 104 to be used throughout the procedure. As such, a mother duodenoscope that requires a small-sized cholangioscope, associated electronics and associated skilled personnel can be eliminated. Thus, without the bulk of a full-functionality duodenoscope, the diameter of elongate body 120 of cholangioscope 104 can be increased, thereby permitting an associated increase in the size of tissue retrieval device 106. Because of the simplicity of guide sheath 102, guide sheath 102 can be configured to be disposable.

FIG. 2 is a schematic diagram of the distal end portions of guide sheath 102, cholangioscope 104, and tissue retrieval device 106 of FIGS. 1A and 1B. Guide sheath 102 can comprise shaft 108 and lumen 136. Cholangioscope 104 can comprise elongate body 120 and lumen 138. Tissue retrieval device 106 is additionally shown extending from lumen 138. Tissue retrieval device 106 can comprise shaft 126 and tissue separator 128. FIG. 2 is not necessarily drawn to scale and may be exaggerated in certain aspects for illustrative purposes.

As illustrated, guide sheath 102, cholangioscope 104 and tissue retrieval device 106 can be configured to be inserted through particular anatomy and nested within each other to maximize the size of tissue retrieval device 106.

Guide sheath 108 can have outer diameter D1 configured for insertion into mouth 201 (FIG. 6) of a patient. Guide sheath 108 can be configured to be as large as anatomy of typical patients will accept, such as esophagus 206 (FIG. 6) and duodenum 202 (FIG. 6). Additionally, guide sheath 108 can be configured in different sizes to be compatible with human anatomy of different sizes. In examples, D1 can be in the range of approximately 10.0 mm to approximately 12.0 mm. In additional examples, D1 can be in the range of approximately 8.0 mm to approximately 9.0 mm.

Lumen 136 of guide sheath 108 can have diameter D2 and can be configured to be as large as feasibly possibly taking into account the desired steerability of guide sheath 108, including flexibility, durability and rigidity. Shaft 108 can have a thickness between D1 and D2 that is adequate to incorporate pull wires 140A and 140B and other associated components, such as pull rings. Control knob 114 (FIG. 1A) can be configured to apply tension to pull wires 140A and 140B to apply a bending force to shaft 108 to, for example, steer guide sheath 102 and apply an associated bending force to cholangioscope 104 when cholangioscope 104 is inserted within lumen 136. However, in other examples, anchor wires can be included inside lumen 136. In examples, given the simple construction of guide sheath 108, guide sheath 108 can be configured as a disposable item intended for single use. In examples, D2 can be in the range of approximately 9.0 mm to approximately 11.00 mm. In additional examples, D2 can be in the range of approximately 8.0 mm to approximately 9.00 mm.

Cholangioscope 104 can have outer diameter D3 configured for insertion into lumen 136 of guide sheath 102. Cholangioscope 104 need not be configured to maximize size available from diameter D2, but can be configured to utilize as much of the space of lumen 136 as is necessary to provide a cholangioscope of desired capabilities. Elongate body 120 can be configured to freely slide within lumen 136 without binding. In examples, D3 can be in the range of approximately 8.0 mm to approximately 10.0 mm. In additional examples, D3 can be in the range of approximately 4.0 mm to approximately 4.5 mm.

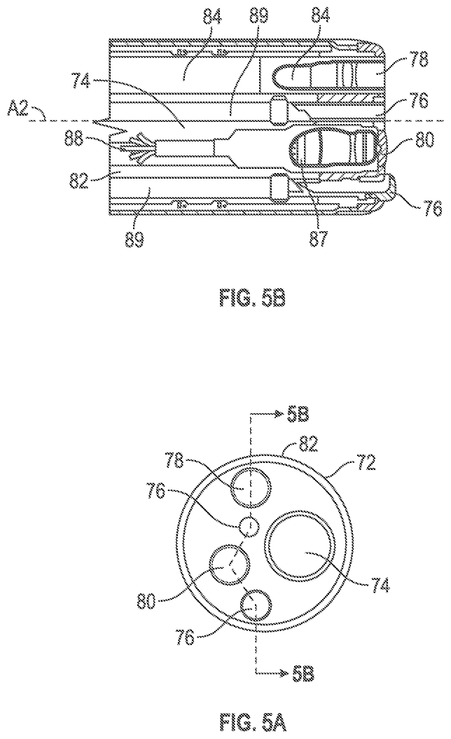

Lumen 138 of cholangioscope 104 can have diameter D4 and can be configured to be as large as feasibly possible taking into account the amount of space required for optical components and other surgical components of cholangioscope 104. As such, lumen 138 can be configured to have one or more lumens 142 for other desired functionality of cholangioscope 104. Lumen 142 can be configured to receive components discussed with reference to FIGS. 5A and 5B, such as light transmitter 84, wiring 88 and fluid lines 89. Likewise, lumen 138 can be configured to accommodate pull wires 146A and 146B, which can be coupled to knob 38 (FIG. 3) in elongate body 120. In examples, D4 can be in the range of approximately 5.0 mm to approximately 6.0 mm. In additional examples, D4 can be in the range of approximately 2.0 mm to approximately 3.0 mm.

FIG. 3 is a schematic diagram of endoscopy system 10 comprising imaging and control system 12 and endoscope 14. The system of FIG. 3 is an illustrative example of an endoscopy system suitable for use with the systems, devices and methods described herein, such as direct peroral cholangioscope systems that can be used for obtaining samples of tissue or other biological matter to be removed from a patient for analysis or treatment of the patient. According to some examples, endoscope 14 can comprise cholangioscope 104 of FIGS. 1A-2 and can be insertable into an anatomical region for imaging and/or to provide passage of one or more sampling devices for biopsies, or one or more therapeutic devices for treatment of a disease state associated with the anatomical region. Endoscope 14 can, in advantageous aspects, interface with and connect to imaging and control system 12. In the illustrated example, endoscope 14 comprises an end-viewing cholangioscope, though other types of endoscopes can be used with the features and teachings of the present disclosure.

Imaging and control system 12 can comprise control unit 16, output unit 18, input unit 20, light source unit 22, fluid source 24 and suction pump 26.

Imaging and control system 12 can include various ports for coupling with endoscopy system 10. For example, control unit 16 can include a data input/output port for receiving data from and communicating data to endoscope 14. Light source unit 22 can include an output port for transmitting light to endoscope 14, such as via a fiber optic link. Fluid source 24 can include a port for transmitting fluid to endoscope 14. Fluid source 24 can comprise a pump and a tank of fluid or can be connected to an external tank, vessel or storage unit. Suction pump 26 can comprise a port used to draw a vacuum from endoscope 14 to generate suction, such as for withdrawing fluid from the anatomical region into which endoscope 14 is inserted. Output unit 18 and input unit 20 can be used by an operator of endoscopy system 10 to control functions of endoscopy system 10 and view output of endoscope 14. Control unit 16 can additionally be used to generate signals or other outputs from treating the anatomical region into which endoscope 14 is inserted. In examples, control unit 16 can generate electrical output, acoustic output, a fluid output and the like for treating the anatomical region with, for example, cauterizing, cutting, freezing and the like.

Endoscope 14 can comprise insertion section 28, functional section 30 and handle section 32, which can be coupled to cable section 34 and coupler section 36.

Insertion section 28 can extend distally from handle section 32 and cable section 34 can extend proximally from handle section 32. Insertion section 28 can be elongate and include a bending section, and a distal end to which functional section 30 can be attached. The bending section can be controllable (e.g., by pull wires 146A and 146B connected to control knob 38 on handle section 32) to maneuver the distal end through tortuous anatomical passageways (e.g., stomach, duodenum, kidney, ureter, etc.). Insertion section 28 can also include one or more working channels (e.g., an internal lumen) that can be elongate and support insertion of one or more therapeutic tools of functional section 30, such as tissue retrieval device 106 of FIGS. 1A and 1B. The working channel can extend between handle section 32 and functional section 30. Additional functionalities, such as fluid passages, anchor wires, and pull wires can also be provided by insertion section 28 (e.g., via suction or irrigation passageways, and the like).

Handle section 32 can comprise knob 38 as well as ports 40. Knob 38 can be coupled to pull wires 146A and 146B, or other actuation mechanisms, extending through insertion section 28 so that rotation of knob 38 can cause bending of functional section 30. Ports 40 can be configured to couple various electrical cables, anchor wires, auxiliary scopes, tissue collection devices of the present disclosure, fluid tubes and the like to handle section 32 for coupling with insertion section 28. For example, cholangioscope 104 can be fed into endoscope 14 via one of ports 40.

Imaging and control system 12, according to examples, can be provided on a mobile platform (e.g., cart 41) with shelves for housing light source unit 22, suction pump 26, image processing unit 42 (FIG. 4), etc. Alternatively, several components of imaging and control system 12 shown in FIGS. 3 and 4 can be provided directly on endoscope 14 so as to make the endoscope “self-contained.”

Functional section 30 can comprise components for treating and diagnosing anatomy of a patient. Functional section 30 can comprise an imaging device, an illumination device and an elevator, such as is described further with reference to FIGS. 5A and 5B. Functional section 30 can comprise imaging and illuminating components configured for end-viewing, e.g., viewing distally or axially beyond of functional section 30.

FIG. 4 is a schematic diagram of endoscopy system 10 of FIG. 3 comprising imaging and control system 12 and endoscope 14. FIG. 4 schematically illustrates components of imaging and control system 12 coupled to endoscope 14, which in the illustrated example comprises an end-viewing cholangioscope. Imaging and control system 12 can comprise control unit 16, which can include or be coupled to image processing unit 42, treatment generator 44 and drive unit 46, as well as light source unit 22, input unit 20 and output unit 18.

Image processing unit 42 and light source unit 22 can each interface with endoscope 14 (e.g., at functional unit 30) by wired or wireless electrical connections. Imaging and control system 12 can accordingly illuminate an anatomical region, collect signals representing the anatomical region, process signals representing the anatomical region, and display images representing the anatomical region on display unit 18. Imaging and control system 12 can include light source unit 22 to illuminate the anatomical region using light of desired spectrum (e.g., broadband white light, narrow-band imaging using preferred electromagnetic wavelengths, and the like). Imaging and control system 12 can connect (e.g., via an endoscope connector) to endoscope 14 for signal transmission (e.g., light output from light source, video signals from imaging system in the distal end, diagnostic and sensor signals from a diagnostic device, and the like).

Fluid source 24 (FIG. 1) can be in communication with control unit 16 and can comprise one or more sources of air, saline or other fluids, as well as associated fluid pathways (e.g., air channels, irrigation channels, suction channels) and connectors (barb fittings, fluid seals, valves and the like). Fluid source 24 can be utilized as an activation energy for a biasing device or a pressure-applying device of the present disclosure. Imaging and control system 12 can also include drive unit 46, which can be an optional component. Drive unit 46 can comprise a motorized drive for advancing a distal section of endoscope 14, as described in at least PCT Pub. No. WO 2011/140118 A1 to Frassica et al., titled “Rotate-to-Advance Catheterization System,” which is hereby incorporated in its entirety by this reference.

FIGS. 5A and 5B illustrate an example of functional section 30 of cholangioscope 14 of FIG. 4. FIG. 5A illustrates an end view of functional section 30 and FIG. 5B illustrates a cross-sectional view of functional section 30 taken along section plane 5B-5B of FIG. 5A. FIGS. 5A and 5B each illustrate “end-viewing endoscope” (e.g., gastroscope, colonoscope, cholangioscope, etc.) camera module 70. In end-viewing endoscope camera module 70, illumination and imaging systems are positioned such that the viewing angle of the imaging system corresponds to a target anatomy located adjacent an end of endoscope 14 and in line with central longitudinal axis A2 of endoscope 14.

In the example of FIGS. 5A and 5B, end-viewing endoscope camera module 70 can comprise housing 72, therapy unit 74, fluid outlets 76, illumination lens 78 and objective lens 80. Housing 72 can comprise and endcap for insertion section 28, thereby providing a seal to lumen 82.

As can be seen in FIG. 5B, insertion section 28 can comprise lumen 82 through which various components can be extended to connect functional section 30 with handle section 32 (FIG. 4). For example, illumination lens 78 can be connected to light transmitter 84, which can comprise a fiber optic cable or cable bundle extending to light source unit 22 (FIG. 4). Likewise, objective lens 80 can be coupled to imaging unit 87, which can be coupled to wiring 88. Also, fluid outlets 76 can be coupled to fluid lines 89, which can comprise a tube extending to fluid source 24 (FIG. 4). In examples, one of fluid outlets 76 can comprise an inlet connected to a fluid line 89 configured for suction, such as being connected to a vacuum, for recovery of lavage and irrigation fluid. Other elongate elements, e.g., tubes, wires, cables, can extend through lumen 82 to connect functional section 30 with components of endoscopy system 10, such as suction pump 26 (FIG. 4) and treatment generator 44 (FIG. 4). For example, therapy unit 74 can comprise a wide-diameter lumen for receiving other treatment components, such as cutting devices and therapeutic devices including tissue retrieval device 106.

Endoscope camera module 70 can also include a photosensitive element, such as a charge-coupled device (“CCD” sensor) or a complementary metal-oxide semiconductor (“CMOS”) sensor. In either example, imaging unit 87 can be coupled (e.g., via wired or wireless connections) to image processing unit 42 (FIG. 4) to transmit signals from the photosensitive element representing images (e.g., video signals) to image processing unit 42, in turn to be displayed on a display such as output unit 18. In various examples, imaging and control system 12 and imaging unit 87 can be configured to provide outputs at desired resolution (e.g., at least 480p, at least 720p, at least 1080p, at least 4K UHD, etc.) suitable for endoscopy procedures.

FIG. 6 is a diagram illustrating guide sheath 102 and cholangioscope 104 of FIGS. 1A and 1B inserted perorally into patient 200 to reach duodenum 202. Guide sheath 102 can extend into mouth 204, through esophagus 206, through stomach 208 to reach duodenum 202. Before reaching intestines 210, guide sheath 102 can position cholangioscope 104 proximate common bile duct 212. Cholangioscope 104 can extend from guide sheath 102 to extend into common bile duct 212. Steering features of guide sheath 102, e.g., pull wires 40A and 40B can be used to facilitate navigating and bending of cholangioscope 104 through stomach 208, in addition to direct steering of cholangioscope 104 via pull wires 146A and 146B. For example, navigation of the Pyloric canal and Pyloric sphincter can be difficult to navigate using only an endoscope. Thus, guide sheath 102 can be used to turn or bend elongate body 120 of cholangioscope 104, or reduce the amount of steering or bending of elongate body 120 required by pull wires 146A and 146B, to facilitate traversing the Pyloric sphincter. Furthermore, once navigated through the Pyloric sphincter, another turn in the opposite direction is typically in order to enter duodenum 202. Again, the steering capabilities of guide sheath 102, which can be relaxed after navigating the pyloric sphincter, can again be employed to turn or bend elongate body 120 of cholangioscope 104 to reduce the burden on the native steering capabilities of cholangioscope 104. In view of the enhanced steering provided by guide sheath 102, the native steering capabilities of cholangioscope 104 (e.g., pull wires 146A and 146B) can be preserved for steering of elongate body 120 to direct tissue retrieval device 106 within duodenum 106.

FIG. 7 is a schematic illustration of a distal portion of guide sheath 102 having cholangioscope 104 and tissue collection device 106 extending therefrom according to the present disclosure. Guide sheath 102 can be positioned in duodenum 202, such as is described with reference to FIG. 6.

Duodenum 202 can comprise duct wall 214, sphincter of Oddi 216, common bile duct 212 and main pancreatic duct 218. Duodenum 202 comprises an upper part of the small intestine. Common bile duct 212 carries bile from the gallbladder and liver (not illustrated) and empties the bile into the duodenum 202 through sphincter of Oddi 216 via passage 220. Main pancreatic duct 218 carries pancreatic juice from the exocrine pancreas (not illustrated) to common bile duct 212. Sometimes it can be desirable to remove biological matter, e.g., tissue, from common bile duct 212 or pancreatic duct 218 to analyze the tissue to, for example, diagnose diseases or maladies of the patient such as cancer.

Cholangioscope 104 can be guided using guide sheath 102 such that a distal end of cholangioscope 104 is positioned proximate sphincter of Oddi 216. Therefrom, a surgeon can operate direct peroral cholangioscopy system 100 to extend tissue retrieval device 106 from cholangioscope 104 to enter common bile duct 212. The native steering capabilities of cholangioscope 104 can be used to turn cholangioscope 104, which in cases can be approximately ninety degrees, to face sphincter of Oddi 216. In examples, cholangioscope 104 can be advanced into common bile duct 212. In either case, tissue retrieval device 106 can be advanced form cholangioscope 104 and manipulated, such as by articulation of cholangioscope 104 using native steering capabilities, to gather target tissue within common bile duct 212. In particular, cholangioscope 104 can be used to navigate tissue retrieval device 106 toward the gall bladder, liver or other locations in the gastrointestinal system to perform various procedures. The surgeon can navigate tissue retrieval device 106 past entry 222 of main pancreatic duct 218 and into passage 220 of common bile duct 212, or into entry 222. Imaging capabilities of cholangioscope (e.g., a camera) can be used to view tissue retrieval device 106 to facilitate direct engagement with the target tissue.

In examples, lumen 138 of cholangioscope 104 can be used to deliver other devices to duodenum 202 and common bile duct 212 instead of or in addition to tissue retrieval device 106. The other devices can have their own functional capabilities, such as a light source, accessories, and biopsy channel, for therapeutic procedures.

Biological matter collected with tissue retrieval device 106 can be removed from the patient, typically by removal of tissue retrieval device 106 from cholangioscope 104, so that the removed biological matter can be analyzed to diagnose one or more conditions of the patient. According to several examples, tissue retrieval device 106 can be suitable for the removal of cancerous or pre-cancerous matter (e.g., carcinoma, sarcoma, myeloma, leukemia, lymphoma and the like), endometriosis evaluation, biliary ductal biopsies, and the like.

As discussed herein, the size of a typical tissue retrieval device is limited by the size of the auxiliary scope, which is itself limited by the size of a duodenoscope. As such, a typical tissue retrieval device can be on the order of approximately 1.2 mm or less. However, with the devices of the present disclosure, guide sheath 102 can be configured as a simple device such that the thickness of guide sheath can be small so that the working channel, e.g., lumen 136 can be large compared to that of a duodenoscope, which must additionally provide passageway for imaging, illuminating and fluid capabilities. As such, with conventional devices, it can be difficult to obtain sufficiently large tissue sample sized to ensure accurate diagnoses without having to repeatedly remove and reinsert the additional device. However, with the systems and devices of the present disclosure it is possible to obtain sufficiently large tissue sample sizes with only a single insertion and removal of the additional device (e.g., tissue retrieval device 106) due to, for example, lumen 138 being enlarged due to the small size of guide sheath 102.

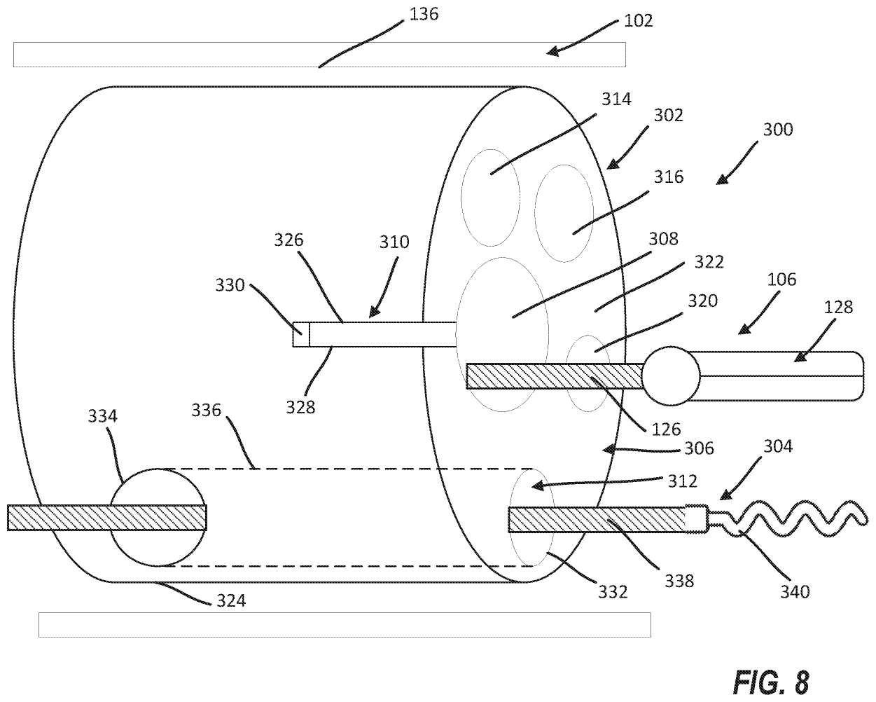

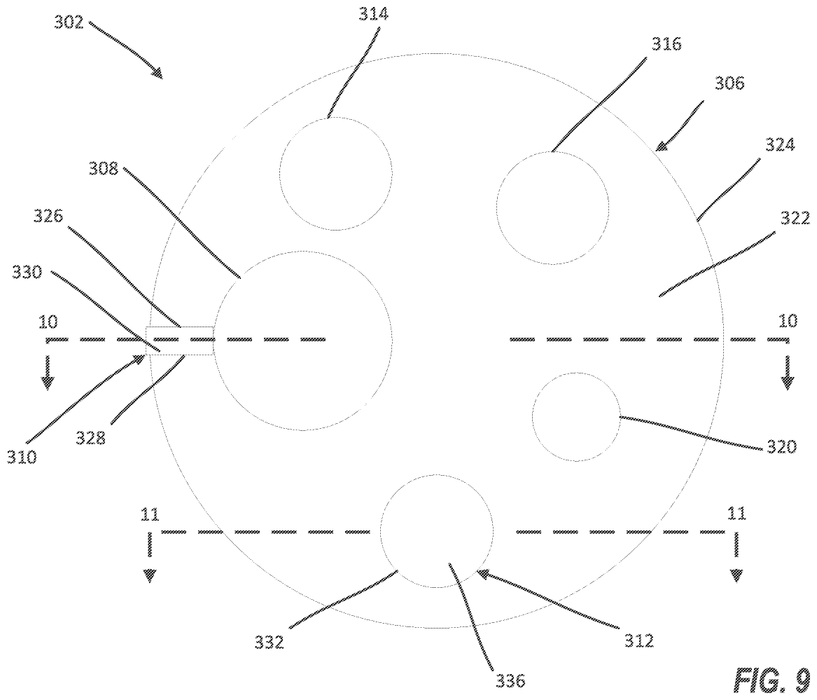

FIG. 8 is perspective view of cholangioscopy system 300 comprising cholangioscope 302 and anchor wire 304 configured for use with guide sheath 102 of the present disclosure. FIG. 9 is a distal end view of cholangioscope 302 of FIG. 8 showing anchor wire lumen 312 and non-axial lumen 310 for working tool lumen 308. FIGS. 8 and 9 are discussed concurrently.

As is discussed in greater detail below, anchor wire 304 can be used with guide sheath 102 to repeatedly insert cholangioscope 302 into anatomy of a patient in order to take multiple tissue samples. In particular, after initial guidance of cholangioscope 302 to the desired target tissue using guide sheath 102, anchor wire 304 can be used to anchor cholangioscope 302 to the target tissue or to tissue proximate the target tissue. Tissue retrieval device 106 can be inserted into working tool lumen 308 to obtain tissue from the patient. Cholangioscope 302 and tissue retrieval device 106 can be withdrawn from the target tissue site, with anchor wire 304 and guide sheath 102 remaining in place. After the target tissue sample is removed from tissue retrieval device 106, cholangioscope 302 can be easily guided back to the target tissue site using guide sheath 102 and anchor wire 304.

Similar to cholangioscope 104 of FIGS. 1A and 1B, cholangioscope 302 can be positioned within guide sheath 102 for steering through anatomy. Cholangioscope 302 can comprise elongate shaft 306, working tool lumen 308, non-axial lumen 310, anchor wire lumen 312, objective lens 314, illumination lens 316, fluid outlet 320, distal end surface 322 and side surface 324.

Objective lens 314 can be configured similarly as objective lens 80 of FIGS. 5A and 5B. Objective lens 314 can be configured to direct light toward an imaging unit to provide digital images to output unit 18. Illumination lens 316 can be configured similarly as illumination lens 78 of FIGS. 5A and 5B. Illumination lens 316 can be configured to direct light from a light transmitter, such as a light transmitter that receives light from light source unit 22, toward tissue distal of distal end surface 322, thereby illuminating tissue for navigation and tissue retrieval device 106. Fluid outlet 320 can be configured similarly as fluid outlets 76 of FIGS. 5A and 5B. One or more fluid outlets 320 can be provided to deliver and recover fluids, such as by being coupled to a fluid source or a suction source. Elongate shaft 306 of cholangioscope 302 can additionally be provided with steering capabilities as is described with reference to cholangioscope 104. For example, elongate shaft 306 can include pull wires such as pull wires 146A and 146B (FIG. 2) that can be coupled to an actuation device to impart curvature to elongate shaft 306.

Working tool lumen 308 can be configured similarly to lumen 138 of endoscope 104 and can be configured to receive a working tool such as tissue retrieval device 106. Working tool lumen 308 can extend from distal end surface 322 to a proximal portion of elongate shaft 306. For example, a proximal end of lumen 308 can be coupled to a port 40 (FIG. 3) configured to allow a working tool to enter elongate shaft 306. The cross-sectional area or diameter of working tool lumen 308 can be sized to allow for tissue separator 128 to pass freely therethrough.

Non-axial lumen 310 can be connected to working tool lumen 308. Non-axial lumen 210 can comprise superior surface 326, inferior surface 328, and proximal surface 330. Anchor wire lumen 312 can comprise distal opening 332, proximal opening 334 and shaft 336.

Non-axial lumen 310 can comprise slot or slit at distal end face 322 of elongate shaft 306 that connects working tool lumen 308 to side surface 324. In examples, non-axial lumen 310 can be rectangular in shape such that proximal surface 330 is flat or planar. Non-axial lumen 310 can be as thick or tall (with reference to the orientation of FIG. 8) as shaft 126 of tissue retrieval device 106 and can be as wide as the thickness between side surface 324 and working tool lumen 308. In examples, non-axial lumen 310 can be as tall as tissue separator 128 to allow tissue separator 128 to pass sideways through non-axial lumen 310. As such, non-axial lumen 310 can extend through material of elongate shaft 306, as well as any coatings, reinforcing layers and the like extending through or along elongate shaft 306. Non-axial lumen 310 can extend along only a distal portion of elongate shaft 306, such as the distal-most ten percent of elongate shaft starting from distal end face 322 and moving proximally. In examples, non-axial lumen 310 can extend from approximately 2.0 mm to approximately 10.0 cm from distal end face 332.

Anchor wire 304 can comprise cable 338 and anchor 340. In the illustrated example, anchor 328 can comprise a screw configured to be inserted into tissue of the patient proximate where tissue retrieval device 106 is desired to retrieve target tissue. However, anchor 340 can comprise any suitable device for attaching to soft tissue or lodging within or against soft tissue, such as a balloon. In other examples, anchor wire 304 can be used without anchor 340 and cable 338 can be blunted, such as be having a ball connected thereto.

Cable 338 can comprise a wire or wire rope over which cholangioscope can be slid. Cable 338 can comprise one or more strands of metallic or polymer material sufficiently strong to be pushed through anatomy and allow other devices to ride along the length of anchor wire 304. In examples, cable 338 can be coated with coatings to facilitate sliding against other components and tissue. In examples, cable 338 can simply comprise a long and skinny member that can be inserted into the anatomy after endoscope 302 is emplaced, such as by being pulled along with cholangioscope 302 during the insertion process. In such configurations, anchor 340 can be configured to catch on opening 332 to facilitate being pulled by cholangioscope 302. However, in other examples, cable 338 can be configured as a steerable anchor wire to be separately navigated to the target tissue, either before or after cholangioscope 302 is navigated to the target tissue site. Thus, anchor wire 304 can be navigated to the target tissue and cholangioscope 302 can follow by inserting a proximal end of cable 338 into opening 332, or cholangioscope 302 can be navigated to the target tissue and anchor wire 304 can follow by being navigated to opening 334.

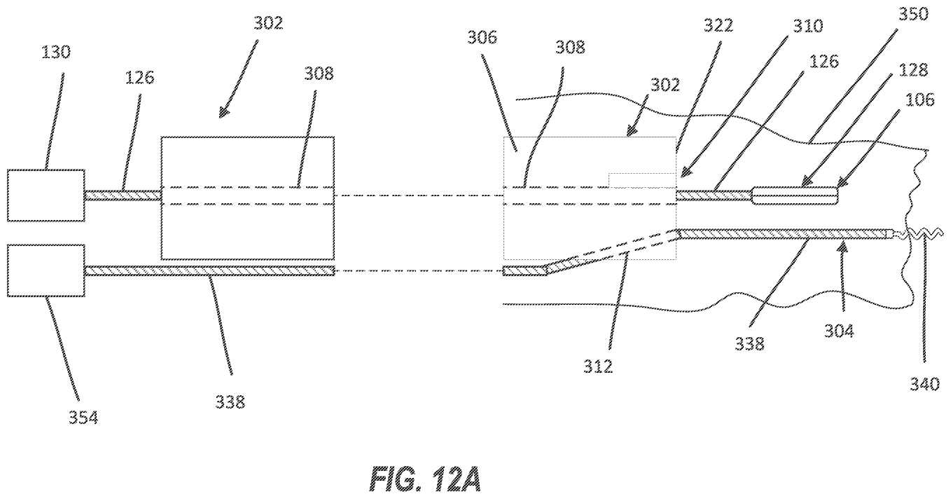

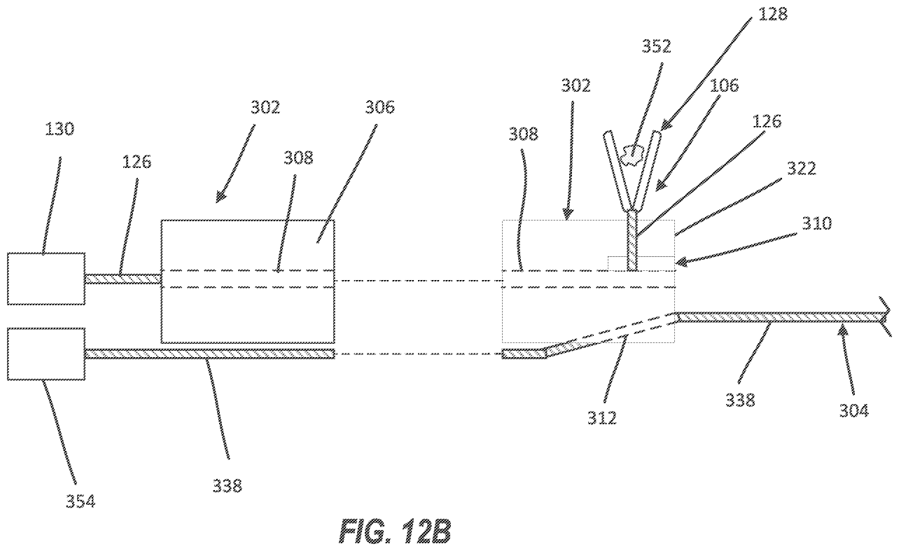

In examples, cable 338 can be connected to controller 354 (FIG. 12B) to facilitate operation of anchor wire 304. For example, controller 354 can include a mechanism to control advancing and retreating of cable 338, such as a thumbwheel. Additionally, controller 354 can be used to deploy or operate anchor 340. For example, controller 354 can apply rotation to cable 338 to allow a corkscrew anchor to penetrate tissue or can provide inflation pressure, via air or fluid pressure, to a balloon.

Tissue retrieval device 106 can be positioned within working tool lumen 308 for insertion of cholangioscope 302 into the anatomy and withdrawal of cholangioscope 302. As discussed herein, tissue separator 128 can be configured as forceps or any other device suitable for separating, retrieving or collecting sample biological matter. Shaft 126 of tissue retrieval device 106 can comprise a pliable body that can allow tissue separator 128 to be angled out of elongate shaft 306 via bending through non-axial lumen 310. Shaft 126 can additionally accommodate passage of control features, such as actuation wires, to tissue separator 128 to facilitate actuation of tissue separator 128 to collect tissue.

FIG. 10 is a schematic cross-sectional view taken along the plane 10-10 of cholangioscope 302 in FIG. 9 to show non-axial lumen 310 connected to working tool lumen 308. Cholangioscope 302 can comprise elongate shaft 306, distal end face 322 and side surface 324. Non-axial lumen 310 can comprise superior surface 326 (FIG. 10), inferior surface 328 and proximal surface 330. Working tool lumen 308 can extend from proximally within elongate shaft 306 to distal end face 322. As shown in FIG. 9, working tool lumen 308 can comprise a circular lumen, however other shapes and cross-sections can be use. Also shown in FIG. 9, non-axial lumen 310 can comprise a rectangular shape or cross-section in the axial direction of elongate shaft 306. As shown in FIG. 10, non-axial lumen 310 can comprise a rectangular shape or cross-section in a direction transvers to the axial direction of elongate shaft 306, as is indicated by the flatness of proximal surface 330. However, proximal surface 330 can be curved between side surface 324 and working tool lumen 308, as shown by line 356, or angled, as shown by line 358, so that non-axial lumen 310 can avoid having sharp surfaces against which shaft 126 of tissue retrieval device 106 is configured to bend.

Non-axial lumen 310 can connect working tool lumen 308 with both distal end face 322 and side surface 324. Thus, when cholangioscope 302 is withdrawn from the anatomy, shaft 126 of tissue retrieval device 106 can be bent to position tissue separator 128 out of working tool lumen 308.

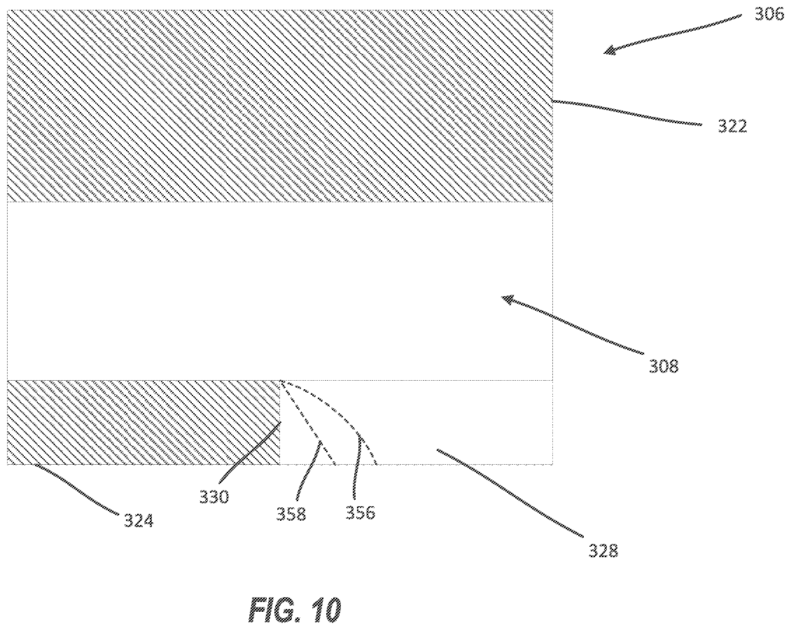

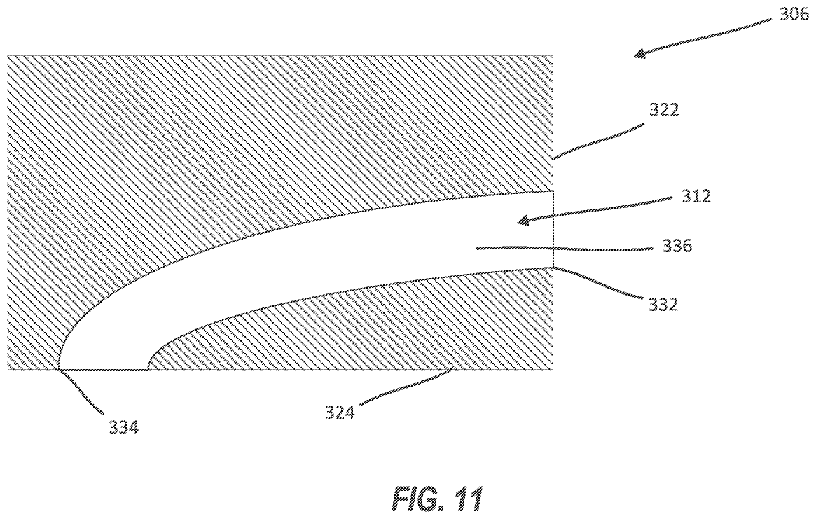

FIG. 11 is a schematic cross-sectional view taken along the plane 11-11 of cholangioscope 302 in FIG. 9 to show anchor wire lumen 312. Cholangioscope 302 can comprise elongate shaft 306, distal end face 322 and side surface 324. Anchor wire lumen 312 can comprise distal opening 332 and side opening 334.

Anchor wire lumen 312 can comprise a passage through elongate shaft 306 to provide a shortcut from distal end face 322 to side surface 324 distally of the proximal end of elongate shaft 306. Anchor wire lumen 312 can allow an instrument to enter and leave elongate shaft 306 without having to pass through the proximal end portion of elongate shaft 302. Thus, anchor wire lumen 312 does not occupy space within elongate shaft 306 of cholangioscope 302 along the majority of the length of elongate shaft 306. The space within elongate shaft 306 can thus be available for other functional components of cholangioscope 302, such as imaging components, illumination components and fluid components. However, anchor wire lumen 312 can be long enough to provide a secure attachment to cable 338 of anchor wire 304 to allow guiding of the distal portion of elongate shaft 306 along the path of anchor wire 304. In examples, anchor wire lumen 312 can be short to provide a “point” connection. However, anchor wire lumen 312 can have a length to facilitate aligning of the axis of elongate shaft 306 with the axis of cable 338.

In the illustrated example, anchor wire lumen 312 is configured as passing through material of elongate shaft 306 within the cylindrical footprint of elongate shaft 306. Such a configuration is advantageous in saving space within elongate shaft 306, as mentioned, and providing a low-profile shape to the distal end portion of elongate shaft 306 to avoid snagging on anatomy and guide sheath 102. However, in other examples, anchor wire lumen 312 can be provided outside of the cylindrical footprint of elongate shaft 306, e.g., on the exterior of elongate shaft 306, such as by passing through a hook or clip attached to elongate shaft 306, or passing through a protrusion of material of elongate shaft 306 forming a hook, clip or eyelet.

FIG. 12A is schematic diagram of cholangioscope 302 of FIG. 8 inserted into tissue duct 350, anchor wire 304 attached to tissue duct 350, and tissue retrieval device 106 extended beyond non-axial lumen 310.

Cholangioscope 302 can be guided to tissue duct 350 using the steering capabilities of guide sheath 102 (FIGS. 1A-2) and the native steering capabilities of cholangioscope 302 (e.g., pull wires). In examples, tissue duct 350 can comprise a duodenum, a common bile duct, a pancreatic duct or other tissue ducts. Using controller 354, anchor wire 304 can be inserted into tissue duct 350 before, simultaneously of after cholangioscope 302. Anchor wire 304 can be positioned with cable 338 extending through anchor wire lumen 312 so that a distal end of cable 338 protrudes therefrom. Anchor 340 can thus be positioned distally of distal end face 322 of elongate shaft 306. Controller 354 can be used to engage anchor 340 with tissue to prevent displacement of cable 338.

Tissue retrieval device 106 can be inserted into tissue duct 350 along with cholangioscope 302 or after cholangioscope 302 is positioned therein. Tissue retrieval device 106 can be extended from working tool lumen 308 so that tissue separator 128 protrudes therefrom. Tissue retrieval device 106 can be manipulated by control device 130 to positioned tissue separator 128 at the site of target tissue within tissue duct 350. In examples, tissue retrieval device 106 can be fabricated of transparent or translucent materials that allow light to pass therethrough, such that objective lens 80 can see through tissue retrieval device 106. Thus, opening of separators 134 to obtain tissue will not impede viewing of the target tissue site by an operator. Control device 130 can thus be operated to rotate separators 134 at hinge 132 (FIG. 1A) to remove target tissue from tissue duct 350. After tissue separator 128 obtains tissue, cholangioscope 302 can be withdrawn from tissue duct 350, such as along anchor wire 304.

FIG. 12B is a schematic diagram of cholangioscope 302 of FIG. 8 withdrawn from tissue duct 350, anchor wire 304 remaining engaged with tissue duct 350, and tissue retrieval device 106 bent to extend through non-axial lumen 310 and release tissue sample 352.

With tissue separator 128 holding a tissue sample, tissue retrieval device 106 can be retreated into working tool lumen 308 so that tissue separator 128 is within elongate shaft 306. Thus, tissue separator 128 can be protected during withdrawal of cholangioscope 302. However, cholangioscope 302 can be withdrawn with tissue separator 128 extending from working tool lumen 308.

Cholangioscope 302 can be retreated backward out of tissue duct 350 (to the left in FIG. 12B). Anchor 340 (FIG. 12A) can remain attached to tissue duct 350 so that cable 338 remains extended along the path from the target tissue site in tissue duct 350 to exit the mouth of the patient during a peroral procedure. Handle section 32 (FIG. 3) can be grasped to pull cholangioscope 302 from tissue duct 350. Thus, tissue retrieval device 106 can be pulled along with cholangioscope 302. As cholangioscope 302 is withdrawn from the patient, the proximal-most portion (e.g., handle section 32) becomes displaced farther and farther from the patient. This can make the entry for working tool lumen 308 be positioned away from the operator of cholangioscope 302. Thus, ordinarily to retrieve tissue from tissue separator 128, an operator would need to move to the location of handle section 32 to either fully withdraw tissue retrieval device 106 from cholangioscope 302, which can encompass movement of the operator potentially the distance of cholangioscope 302 and tissue retrieval device 106, or advance tissue retrieval device 106 forward to tissue separator 128 protrudes from working tool lumen 308, which can encompass placing of one hand of the operator at handle section 32 and requiring another hand of the operator to be fully distal of elongate shaft 306 to engage and open tissue separator 128. Either of these options can produce extra steps in the procedure and additional movement of the operator which can be difficult or inconvenient in the confines of an operating room where things are arranged in a compact and careful manner. However, with the present disclosure, non-axial lumen 310 can facilitate accessing tissue retrieval device 106 quickly and easily without requiring reorienting of the position of tissue retrieval device 106 relative to cholangioscope 302 or repositioning of the operator within the operating room. Shaft 126 can be bent to move tissue separator 128 away from elongate shaft 306. Specifically, shaft 126 can be bent to position tissue separator 128 axially alongside side surface 324 and, in some examples, proximal of distal end face 322. In an example, shaft 126 can be bent at approximately ninety degrees. Thus, an operator can remain positioned proximate the patient where cholangioscope 302 is withdrawn from and manipulate tissue separator 128 easily to access sample tissue 352. For example, tissue separator 128 can be simply grasped distal of cholangioscope 302 and pulled proximally through non-axial lumen 310, and then finely manipulated to access sample tissue 352. In an example, controller 130 can be operated to move separators 128 away from each other to release sample tissue 352.

After sample tissue 352 is retrieved, tissue separator 128 can be repositioned distally of cholangioscope 302 and, if desired, retraced into working tool lumen 308. Thereafter, cholangioscope 302 can be moved back into tissue duct 350 via sliding elongate shaft 306 along cable 338 to the target tissue site. Steering capabilities of guide sheath 102 and cholangioscope 302 can be used in conjunction with anchor wire 304 to facilitate movement of cholangioscope 302 to the target tissue site. However, naked or un-aided manipulation of cholangioscope 302 is not needed due to the presence of anchor wire 304. Once at the target tissue site, tissue separator 128 can again be employed to obtain an additional sample of tissue to supplement tissue sample 352. Thus, sufficiently large quantities of tissue can be obtained to perform one or more different types of tissue analysis procedures, without the difficulty of having to renavigate cholangioscope unaided through the duodenum and common bile duct.

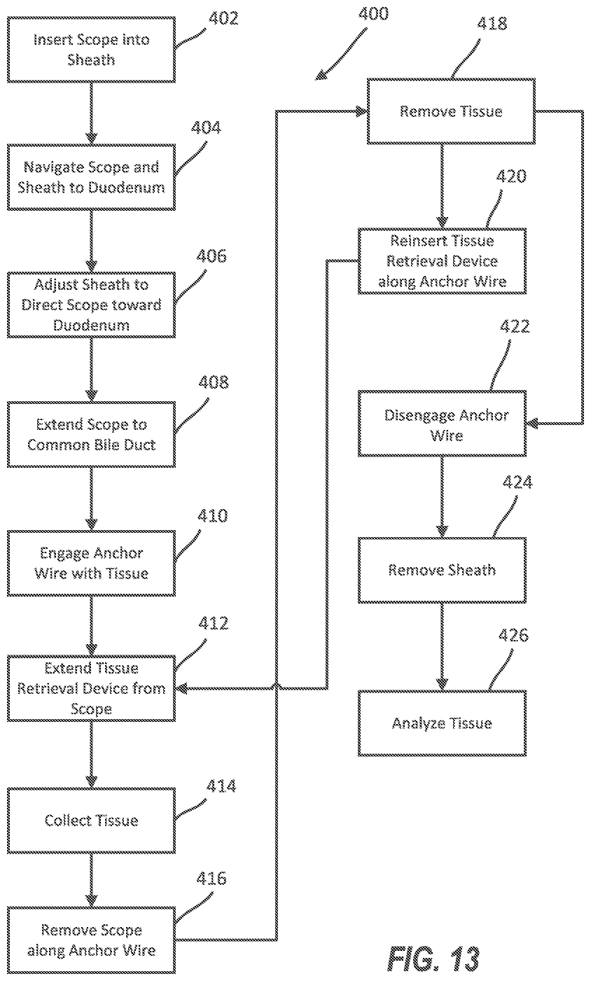

FIG. 13 is a block diagram illustrating examples of method 400 of collecting biological matter from a patient using, for example, direct peroral cholangioscopy system 300 of the present disclosure. Method 400 can encompass the use of guide sheath 102, cholangioscope 302 and tissue retrieval device 106 of FIGS. 8-12B.

At step 402, cholangioscope 302 can be inserted into guide sheath 102. Specifically, elongate shaft 306 of cholangioscope 302 can be inserted into lumen 136 of guide sheath 102. Cholangioscope 104 can be configured to freely slide within guide sheath 102 such that one can move relative to the other during insertion and thereafter.

At step 404, cholangioscope 302 and guide sheath 102 can be navigated through anatomy of a patient. Specifically, cholangioscope 302 and guide sheath 102 can be inserted into mouth 204 of the patient, pushed downward through esophagus 206 to stomach 208 (see FIG. 6). Cholangioscope 302 and guide sheath 102 can be steered to extend through stomach 208 and into duodenum 202.

At step 406, cholangioscope 302 and guide sheath 102 can be pushed into duodenum 202. For example, guide sheath 102 can be steered, such as by using control knob 114 to pull at least one of pull wires 140A and 140B, to bend cholangioscope 302 to exit stomach 208 through the pyloric canal, thereby relieving controls of cholangioscope 302, e.g., pull wires 146A and 146B, from having to apply tension and/or compression to elongate shaft 306, further allowing cholangioscope 302 to have steerability capabilities for further use.

At step 408, guide sheath 102 can be adjusted, such as by using control knob 114 to pull at least one of pull wires 140A and 140B, to orient a distal end of guide sheath 102 toward Sphincter of Oddi 216, which again preserves native steering capabilities of cholangioscope 302, e.g., pull wires 146A and 146B, for later use. However, pull wires 146A and 146B of cholangioscope 302 can supplement action of pull wires 140A and 140B.

Cholangioscope 302 can be steered to duodenum 212 using pull wires 140A and 140B. Rigidity of guide sheath 102 can be used to allow cholangioscope 302 to push off guide sheath 102 to achieve the desired geometry to face towards and enter, if needed, duodenum 212. Cholangioscope 302 can be extended from guide sheath 102 to engage Sphincter of Oddi 216. In other examples, cholangioscope 302 can be extended from guide sheath 102 to penetrate Sphincter of Oddi 216.

At step 410, anchor wire 304 can be extended through anchor wire lumen 312 within elongate shaft 306 of cholangioscope 302. Anchor wire 304 can be extended into cholangioscope 302 before or after cholangioscope 302 is placed in the anatomy. Anchor wire 304 can be extended so that anchor 340 protrudes from anchor wire lumen 312. Anchor 340 can then be deployed to engage tissue. As discussed, anchor 340 can be attached to, adhered to or lodged against tissue to prevent or inhibit proximal displacement of anchor wire 304. Cable 338 can be extended through anchor wire lumen 312 to a proximal end of cholangioscope 302 outside of elongate shaft 306, and can be connected to, for example, handle section 32.

At step 412, tissue retrieval device 106 can be inserted into cholangioscope 302 and extended therefrom. Tissue separator 128 can be extended from the distal end of elongate shaft 306 to engage tissue beyond Sphincter of Oddi 216 within common bile duct 212. In other examples, tissue retrieval device 106 can be guided through stomach 208 and duodenum 202 along with guide sheath 102 and cholangioscope 302.

At step 414, target tissue can be collected using tissue retrieval device 106. The target tissue can comprise tissue that is potentially diseased or otherwise indicative of a diseased condition of the patient. For example, separators 132 can be manipulated from control device 130 to engage target tissue one or more times to collect, separate if necessary, and store target tissue. In examples, tissue retrieval device 106 can be sized large enough, due to the factors discussed herein, to collect a sufficient volume of biological matter in a single collection operation such that multiple insertions of tissue retrieval device 106 can be avoided. However, with the examples of FIGS. 8-12B, tissue retrieval device 106 can be sized to any suitable capacity and can be configured to be easily removed and reinserted into the anatomy without having to re-execute manipulation of guide sheath 102 and cholangioscope 302 to navigate the intricacies of stomach 208 and common bile duct 212. For example, over-sized forceps can be used that partially obstruct the imaging capabilities of cholangioscope 302 when deployed out of working tool lumen 308, particularly if the forceps are fabricated substantially or partially from transparent materials and components.

At step 416, tissue retrieval device 106 can be withdrawn into working tool lumen 308 of cholangioscope 308. Cholangioscope 302 can be withdrawn from the anatomy, by sliding along cable 338, so that target tissue can be removed from tissue separator 128. Anchor 340 can remain attached or engaged with tissue to that anchor wire 304 remains in place within the anatomy.

At step 418, the target tissue can be removed from tissue separator 128. For example, separators 134 can be opened to allow access to the separated target tissue.

Thereafter, method 400 can return to step 412 via reinserting cholangiocope 302 and tissue retrieval device 106 into the anatomy at step 420 to collect additional matter from the previous target tissue site or collect additional matter from a different target tissue site, or can continue to step 422 to complete the operation.

At step 422, anchor 304 can be removed from the patient, such as by disengaging anchor 340 from the tissue and then retracting cable 338 from the anatomy.

At step 424, guide sheath 102 can be removed from the patient, such as by withdrawal from esophagus 206.

At step 426, tissue collected form the patient can be analyzed, such as by being transported to a laboratory to be analyzed for the presence of diseased tissue, such as cancerous tissue.

As such, method 400 illustrates examples of methods of collecting biological matter from internal passages of a patient in multiple samples so that large enough quantities of the tissue can be retrieved during a single patient procedure for performing analysis, thereby reducing or eliminating the need for a follow-on patient procedure at a later time to obtain additional tissue. Method 400 can be performed using a cholangioscope having an anchoring system comprising an anchor wire, an anchor wire lumen and a non-axial lumen for a working tool. The anchor wire and anchor wire lumen can be used to guide the cholangioscope into and out of the anatomy of the patient without having to re-perform intricate insertion and steering maneuvers. The anchor wire can be attached only to a distal portion of the cholangioscope to avoid occupying space within the cholangioscope shaft. The non-axial lumen can be used to easily access a tissue retrieval, collection or separator device at the distal end of a working tool without having to completely withdraw the working tool from the cholangioscope. Thus, direct peroral cholangioscopy system 300 of the present disclosure can facilitate efficient tissue collection from a patient, reducing procedure times and the chances for the need of a follow-on procedure.

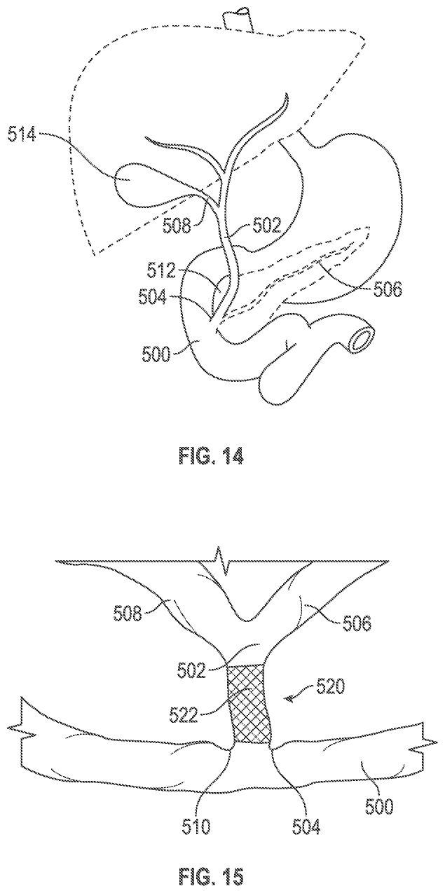

FIG. 14 is a schematic view of duodenum 500 connected to common bile duct 502 via duodenal papilla 504. Common bile duct 502 can branch off into pancreas duct 506 and gallbladder duct 508. Duodenal papilla 504 can include sphincter of Oddi 510 (FIG. 15). Pancreas duct 506 can lead to pancreas 512. Gallbladder duct 508 can lead to gallbladder 514. As discussed above, it can be difficult to navigate surgical instruments to duodenal papilla 504. It can also be difficult to navigate a surgical instrument into common bile duct 502 via insertion through duodenal papilla 504. For example, sphincter of Oddi 510 is a muscle that can control flow of bile and pancreatic juice into the intestine. The muscle of sphincter of Oddi 510 can, however, make it difficult to enter common bile duct 502. It is, therefore, common during medical procedures to cut sphincter of Oddi 510 to enlarge duodenal papilla 504 to allow for easier access of instrument into common bile duct 502.

FIG. 15 is a schematic view of duodenum 500 of FIG. 14 with implant 520 of the present disclosure inserted into duodenal papilla 504. Implant 520 can comprise body 522, which can comprise an annular cylindrical body that pushes sphincter of Oddi 510 into an enlarged state. Implant 520 can be delivered to duodenum 500 in a collapsed state and then enlarged to provide a portal into common bile duct 502. As discussed herein, implant 520 can be used as a foundation to which other components can be mounted to facilitate 1) operation of implant 520, 2) performance of the procedure in which implant 520 is implanted, and 3) performance of a subsequent procedure, among other things.

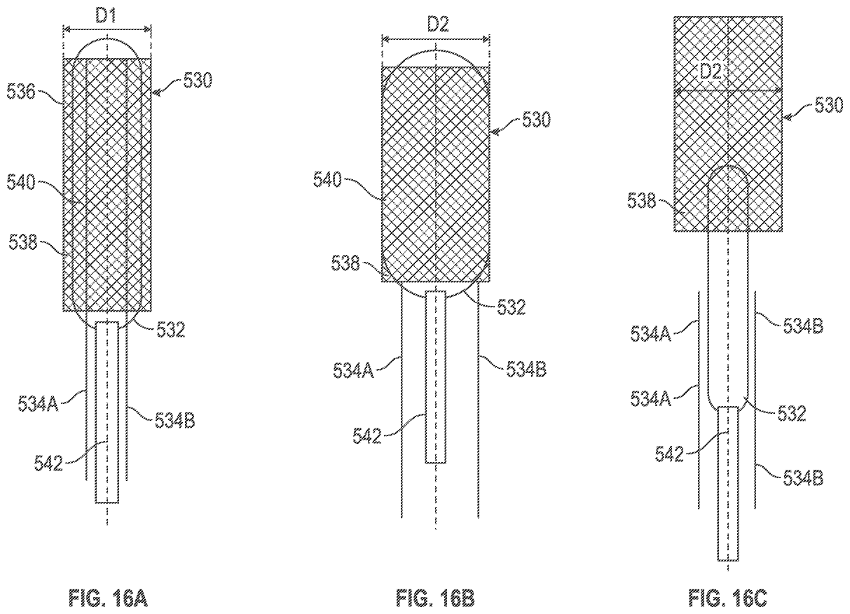

FIG. 16A is a schematic view of stent 530 having inflation balloon 532 inserted therein and lead wires 534A and 534B extending therethrough. FIG. 16A shows stent 530 and balloon 532 in a collapsed state. Stent 530 can comprise a mesh body having outer diameter 536 and internal space 538. Lead wires 534A and 534B can extend from a distal end of stent 530, through the mesh body and can extend proximally from stent 530. Balloon 532 can comprise an inflatable bladder having internal space 540 and tube 542 extending therefrom. Lead wires 534A and 534B and tube 540 can extend through a suitable insertion instrument, tube or sheath that can be used to extend stent 530 and balloon 532 through the working channel of a scope. In examples, stent 530 and balloon 532 can be navigated to duodenum 500 using any of the devices described herein. For example, stent 530 and balloon 532 can be guided through working channel 142 (FIG. 2) of scope 104.

In order to push stent 530 into duodenal papilla 504, sphincter of Oddi 510 (FIG. 2) can be cut to relax the tissue of duodenal papilla 504 to facilitate insertion of stent 530. Lead wires 534A and 534B can be electrified via an electric current to provide cutting of tissue via known electrosurgery techniques, as is discussed below in greater detail with reference to FIG. 16. Lead wires 534A and 534B can be connected to control unit 16 (FIGS. 1A and 4). In examples, a high-frequency alternating current can be used to heat the tissue to a point of cauterization, thereby causing separation from adjacent tissue. Duodenal papilla 504 can be cauterized to reach sphincter of Oddi 510. As such, duodenal papilla 504 to accept stent 530.

FIG. 16B is a schematic view of stent 530 of FIG. 16A in an expanded state with electric lead wires 534A and 534B pulled back from stent 530. Balloon 532 can be inflated to enlarge stent 530 from diameter D1 to diameter D2. Lead wires 534A and 534B can be stiff to hold stent 530 in the collapsed state of FIGS. 2 and 3 during the cutting procedure. However, lead wires 534A and 534B can be withdrawn from stent 530 to allow expansion via inflation of balloon 532. Balloon 532 can be inflated by the passing of pressurized air through tube 542. Balloon 532 can thereby expand internal space 538. Material of stent 530 can stretch or deform to enlarge to the expanded state.

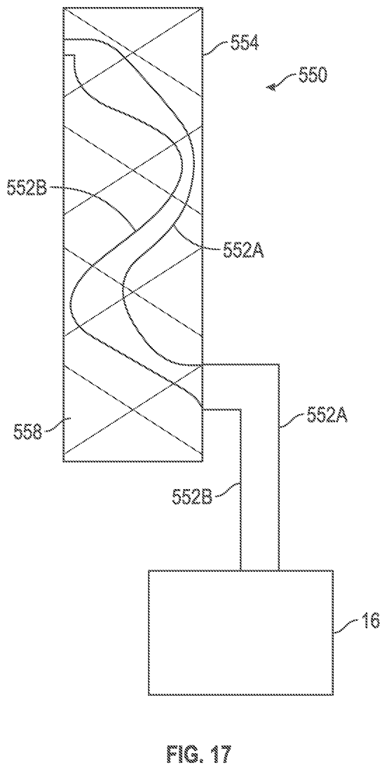

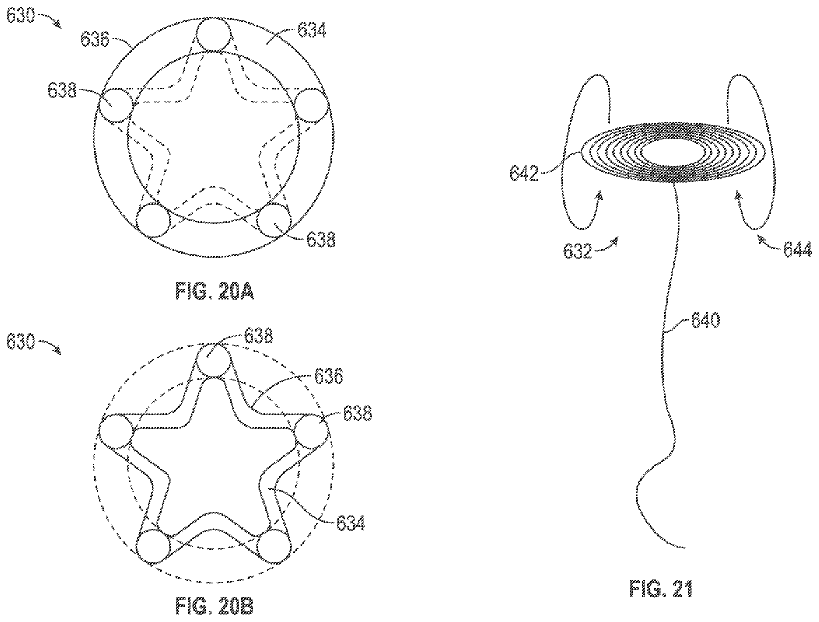

FIG. 16C is a schematic view of stent 530 of FIG. 16B in an expanded state with electric leads 534A and 534B pulled away from stent 530. Material of stent 530 can maintain shape after balloon 532 is deflated. As such internal space 540 can be maintained at diameter D2. Thus, balloon 532 and lead wires 534A and 534B can be withdrawn from stent 530 and the patient through the working channel of the insertion device. As described below with reference to FIGS. 17-19, stents of the present application can be provided with a variety of cutting capabilities. As discussed below with reference to FIGS. 20-39, stents of the present application can be used to facilitate performance of additional procedures at a later point in time such as by providing reentry devices that can expedite steering and/or navigation of instruments back to the anatomy of the stent. Furthermore, the reentry devices can be provided with treatment devices that can perform intermittent, ongoing, or on-demand treatment of the patient between procedures.

FIG. 17 is a schematic side view of stent 550 of the present application including electric leads 552A and 552B extended into cylindrical mesh stent body 554. Electric leads 552A and 552B can be connected to control unit 16 (FIGS. 3 and 4). Stent body 554 can be made up of a plurality of wires 556 interconnected or woven to form internal space 558. Leads 552A and 552B can be interwoven into wires 556. Control unit 16 can be configured to direct various forms of energy to lead wires 552A and 552B to provide various functionality to stent 550. In examples, control unit 16 can provide direct current or alternating electrical current to lead wires 552A and 552B. The electrical energy can be used to cut, coagulate, desiccate, or fulgurate tissue. Furthermore, the electrical energy can be used to provide power to various devices mounted or connected to stent 550.

Lead wires 552A and 552B can comprise conducting wires, bundles of wires or cables that can be attached to body 558. In examples, lead wires 552A and 552B can be formed of copper, copper alloys or other conducting metals and metal alloys. In examples, lead wires 552A and 552B can be made of bioresorbable material that can be naturally disintegrated into the body where stent 550 is deployed. Lead wires 552A and 552B can be attached to body 558 in fixed or deployable manners. For example, lead wires 552A and 552B can be affixed to body 558 for embodiments where lead wires 552A and 552B are intended to remain implanted within anatomy. As disclosed herein, lead wires 552A and 552B can thus be employed to provide treatment to the patient, such as via pumping action or stone ablation action. Lead wires 552A and 552B can thus be welded or soldered to body 558. In additional examples, lead wires 552A and 552B can be woven into the material of body 558. Body 558 can be made of mesh material, such as strands of biocompatible metal, that can be woven into an annular tube that can be radially contracted or expanded. In additional examples, body 558 as well as some or all of the other components of the various devices and stents described herein can be radiopaque to, for example, facilitate viewing in imaging, such as x-ray images and fluoroscopy, to facilitate guiding back to the location of the reentry devices described herein. In embodiments wherein lead wires 552A and 552B are intended to be withdrawn from the patient after use, lead wires 552A and 552B can be releasably attached to body 558. In examples, lead wires 552A and 552B can be configured to be pulled proximally from body 558 out of the anatomy. Thus, lead wires 552A and 552B can be loosed from the woven material of body 558 or welds or soldering attaching lead wires 552A and 552B can be broken to detach lead wires 552A and 552B from body 558. The detached lead wires 552A and 552B can be removed from the anatomy or left therein to biologically dissolve.

Lead wires 552A and 552B can be positioned so that the length of lead wires 552A and 552B on body 558 or a substantial amount of the length of lead wires 552A and 552B on body 558 can contact tissue to provide the desired effect. Lead wires 552A and 552B can be placed in different patterns on body 558, such as straight, undulating and chevron-shaped patterns. Lead wires 552A and 552B can be positioned on the outside of body 558. As mentioned, lead wires 552A and 552B can be used to cut tissue. In particular, lead wires 552A and 552B can be used to cut sphincter of Oddi 510 (FIG. 15) to allow for expansion of stent 550 as disclosed herein. However, in other examples, lead wires 552A and 552B can be used to provide power to other devices or other functionality of stent 550.

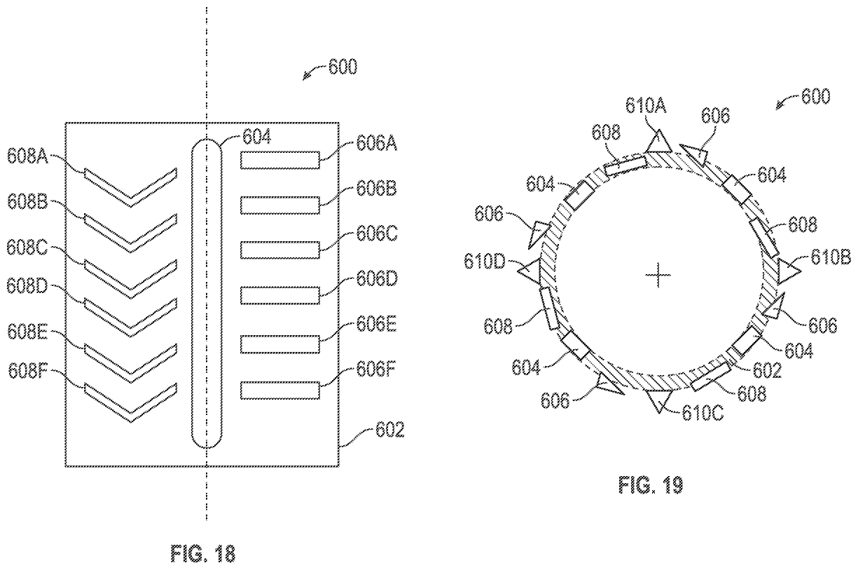

FIG. 18A is a schematic side view of a stent 600 of the present application including a plurality of different mechanical cutting edges disposed in body 602. FIG. 18B is an end view of stent 600 of FIG. 18A. FIGS. 18A and 18B are discussed concurrently.

The mechanical cutting edges can comprise axial edge 604, circumferential edges 606A-606F and angled edges 608A-608F. Stent 600 can further comprise anchors 610A-610D (FIG. 19). Axial edge 604, circumferential edges 606A-606F and angled edges 608A-608F can form a pattern of cutting features on stent 600 that is repeated in the circumferential direction, for example, as can be seen in FIG. 19.

The mechanical cutting edges of stent 600 can be used to cut tissue, such as sphincter of Oddi 510 (FIG. 15) to allow for expansion of stent 600 as disclosed herein.

Axial edge 64 can extend parallel to a central axis of stent 600 and can comprise an edge configured to shave, slice or cut tissue when stent 600 is rotated in a circumferential direction along the central axis. Axial edge 64 can be configured to, for example, cut when rotated in a clockwise direction and not cut when rotated in a counterclockwise direction. Axial edge 64 can thus be formed from a portion of an edge of a cut-out in body 554.

Circumferential edges 606A-606F can extend perpendicular to a central axis of stent 600 and can comprise edges configured to shave, slice or cut tissue when stent 600 is pushed or pulled in an axial direction. Circumferential edges 606A-606F can be configured to, for example, cut when pulled proximally and not cut when pushed distally. Circumferential edges 606A-606F can thus be formed from a portion of an edge of a cut-out in body 554.

Angled edges 608A-608F can extend oblique to a central axis of stent 600 and can comprise edges configured to shave, slice or cut tissue when stent 600 is moved relative to tissue engaged with stent 600. Angled edges 608A-608F can be configured to, for example, cut when rotated in a circumferential direction along the central axis or pulled proximally and not cut when pushed distally. Circumferential edges 606A-606F can thus be formed from a portion of an edge of a cut-out in body 554.

In examples, axial edge 604, circumferential edges 606A-606F and angled edges 608A-608F can be formed in metal plates attached to the mesh of body 602.

FIG. 19 shows stent 600 comprising barbs 610A-610D. Barbs 610A-610D can comprise anchoring features to hold stent 600 in place once disposed. Barbs 610A-610D can be located at one end of body 602 to allow engagement with tissue after mechanical cutting edges are employed. For example, barbs 610A-610D can be located at a proximal end of body 602 to allow distal portions of stent 600 to cut tissue via axial displacement before barbs 610A-610D take hold. In other examples barbs 610A-610D can be axial aligned along body 602 instead of circumferentially arranged as shown in FIG. 19 to allow circumferential of body 602 to allow cutting before barbs 610A-610D to take hold.

FIG. 20A is a schematic cross-sectional view of magnetically-activated stent device 630 in a collapsed state. FIG. 20B is a schematic cross-sectional view of magnetically-activated stent device 630 of FIG. 20A in an expanded state. FIG. 21 depicts an example of magnetic applicator 632. FIGS. 20A-21 are discussed concurrently. Device 630 of FIGS. 20A and 20B can comprise an example of stent 520 of FIG. 15 and can include cutting features of FIGS. 16-19.

FIGS. 20A and 20B illustrate schematic diagrams of a device 630, such as a stent or shunt or plug, capable of radial expansion. Device 630 can be removed after a period of time, left in a patient permanently, or may biodegrade within the patient. FIGS. 20A and 20B illustrate a schematic cross-sectional view of device 630 in open and closed positions. FIG. 21 illustrates an applicator 632 for device 630.

In some cases, device 630 can be a stent, shunt, or plug for insertion into a patient passageway. Device 630 can be a stent for maintaining patency of a patient passageway such as to allow fluid and debris flow therethrough. Device 630 can be a shunt such as for an alternative path for the passage of the blood or other body fluid. Device 630 can be a plug such as for preventing flow of fluid through the passageway.

Device 630 can be sized, shaped, or arranged for full or partial insertion into an anatomic duct or opening, such as duodenal papilla 504 of FIG. 15.

Device 630 can comprise deformable elongated tubular body 634 with sheath 636 and a plurality of magnetizable or magnetizable or magnetic elements 638. Device 630 can be placed or actuated with applicator 632, which can include guide wire 640 and magnet 642. Magnet 642 can be configured to generate magnetic field 644.

Tubular body 634 can extend from a proximal portion to a distal portion. The proximal portion can be for holding, securing, or manipulation of device 630, directly or indirectly, such as by a surgeon or doctor using device 630 for a medical procedure. The proximal portion can optionally be connected to one or more grips, handles, or guidewires, as desired for the operator. The distal portion can be configured for at least partial insertion into a body lumen of a patient.

The deformable elongated tubular body 634 can be capable of an expanded state shown in FIG. 20A and a collapsed state as shown in FIG. 20B. In examples, in the expanded state, tubular body 634 can have a diameter of about 0.5 mm to about 2.0 mm. In the expanded state, tubular body 634 can be located against an inner wall of a body passageway such as to help maintain patency of the passageway. In some cases, the expanded state can distend the body passageway, if desired. The expanded state of tubular body 634 can have a diameter that is comparatively larger than some other stents, such as to allow passage of fluid and debris therethrough.

In examples, in the collapsed state, tubular body 634 can have a diameter of about 0.5 mm to about 2.0 mm. In the collapsed state, tubular body 634 can be collapsed in on itself such as to make a shape with a diameter or other lateral dimension that can be smaller than those of conventional or other stents. This can allow for easy insertion by the operator into a passageway of a patient.

Sheath 636 can include a compliant material extending between the distal portion and the proximal portion of tubular body 634, forming the tubular shape and defining a luminal space, such as a longitudinal lumen in tubular body 634. Sheath 636 can be made of a thin-walled polymer, such as polyethylene, silicone, or polyether block amide. In examples, sheath 634 can have a thickness of about 0.051 mm to about 0.254 mm. In some cases, sheath 636 can include more than one layer of material. Sheath 636 can have about a Shore D durometer of about 0.005′ to about 0.04″. Sheath 636 can define a luminal space with a diameter of about 1 mm to about 20 mm when in an expanded state and less than about 2 mm when in a collapsed state. In an example, the ratio of diameters in the expanded and collapsed state can be about 10:1. In an example, the ratio of diameters in the expanded and collapsed state can be about 25:1. In an example, the ratio of diameters in the expanded and collapsed state can be between about 10:1 and about 25:1. When in a collapsed state, device 630 can be inserted into a patient passageway by itself or in a delivery sheath or shell such as can help it maintain its collapsed search such as during insertion.

Magnetizable or magnetic elements 638 can be embedded in, attached to, or coupled with sheath 634. Magnetic materials can include those that exhibit a response to a change in magnetic field, and can include materials that are aligned to a particular magnetic field. Similarly, magnetizable materials can include those that are capable of being magnetized, and can include materials that are not yet magnetized but could be when exposed to a magnetic field.

Magnetizable or magnetic elements 638 can be at least two magnetic or magnetizable elements in tubular body 634. In some cases, elements 638 can be a group of wires that are woven or braided around a core. In some cases, elements 638 can be actuated selectively. When magnetized, or exposed to a magnetic field, magnetizable or magnetic elements 638 can repel each other, forcing sheath 636 to open to the more expanded state. This can allow for sheath 636 to be magnetically disposed outward to the body passageway wall. The magnetic repulsion between magnetizable or magnetic elements 638 can allow for device 630 to be suspended within a passageway and/or facilitate retention of device 630 in a passageway, such as without need for other securing mechanisms, such as “pigtails,” catches, clips, or other components. Instead, the magnetic force can, in some cases, be used to hold the device in place within a passageway and maintain patency.

In some cases, two, three, four, five, or more magnetizable or magnetic elements 638 can be used in device 630. At least two magnetizable or magnetic elements 638 can be used to allow for magnetic repulsion between those elements when actuated. By manipulating the number and placement of wires, the diameter or other lateral dimension of device 630 in the expanded state can be controlled.

In device 630, magnetic elements 638 can be magnetizable or magnetic elements that are elongate members, and can run along the length of tubular body 634 from the distal portion to the proximal portion. The placement of magnetizable or magnetic elements 638 around the circumference or periphery of sheath 636 can allow for radial expansion when magnetizable or magnetic elements 638 are actuated to repel each other. In some cases, the magnetic repulsion can be sufficient to cause conformal contact between device 630 and a wall that defines the body lumen into which it is placed. In this case, the expanded state can have a circular cross-section, or have a non-uniform cross-section depending on the body lumen shape.

Magnetizable or magnetic elements 638 can include wires, pieces of magnetic material, braided or interwoven strands, or other magnetic dipole inducing material. Magnetizable or magnetic elements 638 can be made of a variety of materials, such as magnetizable metallic or composite materials, or one or more combinations thereof. In some cases, magnetizable or magnetic elements 638 can include magnetic dipole elements affixed to sheath 636.

Magnetizable or magnetic elements 638 can run along the length of tubular body 634 in a longitudinal direction for some or all of the length of tubular body 634, can run along tubular body 634 in a radial direction, can spiral around tubular body 634, can be applied in segments along tubular body 634, or one or more combinations thereof. Magnetizable or magnetic elements 638 can additionally be of varying types, materials, and thicknesses, so as to induce various different magnetic fields, magnetic field reactions, and magnetic repulsions, depending on how magnetizable or magnetic elements 638 are actuated.

FIG. 21 depicts an example of magnetic applicator 632. Magnetic applicator 632 can include guide wire 640 and magnet 642, and can be affected by or configured to generate magnetic field 644. Magnetic applicator 632 can be detachable from device 630, fully, or partially integrated with device 630.

Magnetic applicator 630 can include a monolithic piece, or multiple pieces, such as guidewire 640 and magnet 642. Magnetic applicator 632 can include a ring such as configured to fit in or around the longitudinal lumen of tubular body 634, such as magnet 642. In this case, magnet 642 can be shaped to fit inside or outside tubular body 634, and fitted to the collapsed or expanded state of tubular body 634.

Guidewire 640 can be attached to magnet 642 such as to allow for manipulation of the placement of magnet 642 in or near device 630. This can allow for magnet 642 to add to or change a magnetic field interacting with magnetizable or magnetic elements 638, such as when the magnet is moved. For example, magnetic applicator 632 can be drawn through the longitudinal lumen of tubular body 634 to actuate magnetizable or magnetic elements 638 to help provide magnetic repulsion to encourage tubular body 634 to maintain a more expanded state. This can be done by the operator using the guidewire 640 to move magnets 638.

In some cases, magnetic applicator 632 can be drawn through the longitudinal lumen of tubular body 634 to actuate magnetizable or magnetic elements 638 such as to reduce magnetic repulsion to encourage tubular body 634 to form a less expanded state. This can be done by the operator using guidewire 640 to move magnet 642.

In an example, magnetic applicator 632 can be inserted into the passageway of the patient together or concurrently with device 630. Magnetic applicator 630 can then be drawn out of the patient passageway through device 630 to magnetize magnetizable or magnetic elements 638 and move tubular body 634 from a collapsed state to an expanded state.

In this case, when the operator deems it time to remove device 630 from the patient passageway, magnetic applicator 632 can be used to collapse device 630 for easy removal. In some cases, magnetic applicator 632 can remain attached to device 630. In some cases, magnetic applicator 630 can be removed from device 630 during or after insertion of device 630 into a patient passageway. Magnetic applicator 632 can be re-useable or disposable.

In some cases, magnetic applicator 632 can be used to alter the placement or expansion of device 630. In this case, if the operator deems that device 630 should be adjusted, such as by a patient indicating pain or discomfort, magnetic applicator 632 can be inserted back into the patient passageway in or near device 630. Magnetic applicator 632 can be used to collapse part or all of device 630, allowing the operator to re-position the device. Device 630 can then be re-expanded as desired with magnetic applicator 632, once re-positioning has been completed.

In an example, magnetic applicator 632 can be inserted into the passageway of the patient separately from device 630. In this case, the operator can insert device 630 and subsequently insert magnetic applicator 632, drawing it up through the device to magnetize magnetizable or magnetic elements 638 and moving the device from a collapsed state to an expanded state. In some cases, the magnetic applicator can be left in the patient and removed later when the operator is ready to remove device 630 from the patient passageway.

Magnetic applicator 632 can allow for operator manipulation of device 630, during insertion of device 630, to revise or re-position the placement of device 630, during removal of device 630, or combination thereof.

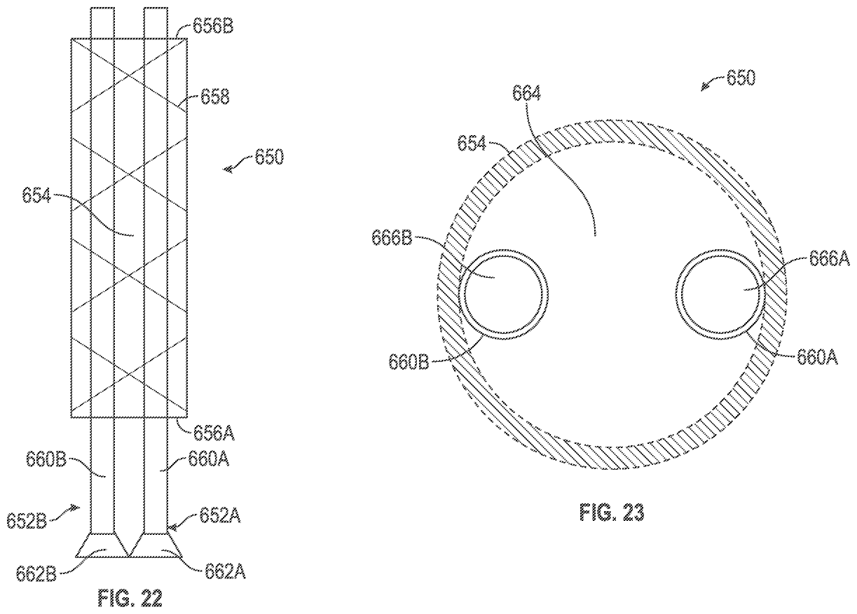

FIG. 22 is schematic side view of stent 650 of the present application including extendable elongate reentry devices 652A and 652B. FIG. 23 is an end view of stent 650 of FIG. 22. FIGS. 22 and 23 are discussed concurrently. Stent 650 can incorporate any of the features and components described with reference to device 630 of FIGS. 20A and 20B, stent 520 of FIG. 15 and can include cutting features of FIGS. 16-19.

Stent 650 can comprise body 654 extending from first end 656A to second end 656B. Body 654 can be made up of wires 658. Reentry device 652A can comprise elongate tube 660A and entry element 662A. Reentry device 652B can comprise elongate tube 660B and entry element 662B. As shown in FIG. 23B, stent 650 can define lumen 664 and tubes 660A and 660B can define lumens 666A and 666B.

Reentry devices 652A and 652B can be configured to help guide another elongate body through stent 650 and into anatomy distal of stent 650 via lumens 666A and 666B. Reentry devices 652A and 652B can comprise flexible bodies that can traverse transitions between intersections of anatomic passageways. In examples, reentry devices 652A and 652B can comprise tubes or guide wires fixedly or slidably attached to body 654. Reentry devices 652A and 652B can extend proximally of body 654 to receive other components or instruments and can extend distally of body 654 to guide the other components or instruments to desired anatomy. In examples, the positions of reentry devices 652A and 652B relative to body 654 can be adjusted. In examples the lengths of reentry devices 652A and 652B can be adjustable.

In examples, reentry devices 652A and 652B can be configured to receive an instrument from duodenum 500, turn the instrument into duodenal papilla 504, direct the instrument through body 654 and guide the instrument into one of pancreas duct 506 and gallbladder duct 508. As such, it can become unnecessary to steer or guide the instrument through such passageway.

Reentry devices 652A and 652B can comprise flexible tubes fabricated from a polymer or another suitable biocompatible material, such as Pebax® [e.g., polyether block amide or PEBA, which is a thermoplastic elastomer (TPE)], nylon, silicon and urethane. Reentry devices 652A and 652B can have a telescopic construction so as to allow distal extension. In additional examples, reentry devices 652A and 652B can be slidably attached to body 654 so that the lengths of reentry devices 652A and 652B extend proximally and distally of body 654 can be adjusted. Thus, reentry devices 652A and 652B can be mounted on rails or can be attached via hoops or clasps. In examples, elongate tubes 660A and 660B can be configured to be released from and removed from body 654 after use to leave stent 650 alone in the anatomy.

The cross-sectional area of lumens 666A and 666B compared to the cross-sectional area of lumen 664 can be small, as shown in FIG. 23. As such, lumen 664 can be free to accept other instruments. However, the ratio of cross-sectional areas of lumens 666A and 666B to lumen 664 can be varied according to performance capabilities for different objectives.

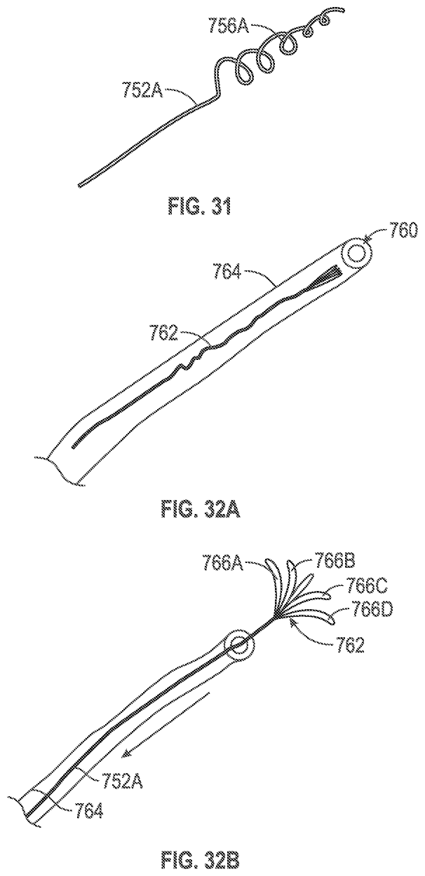

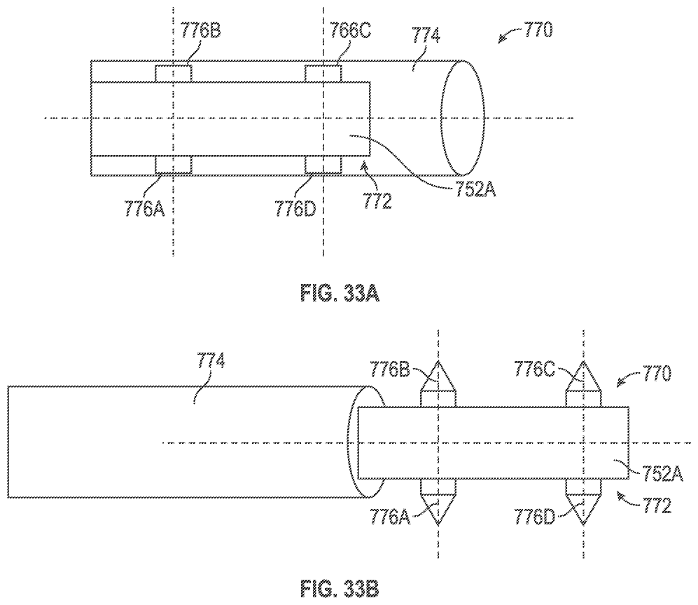

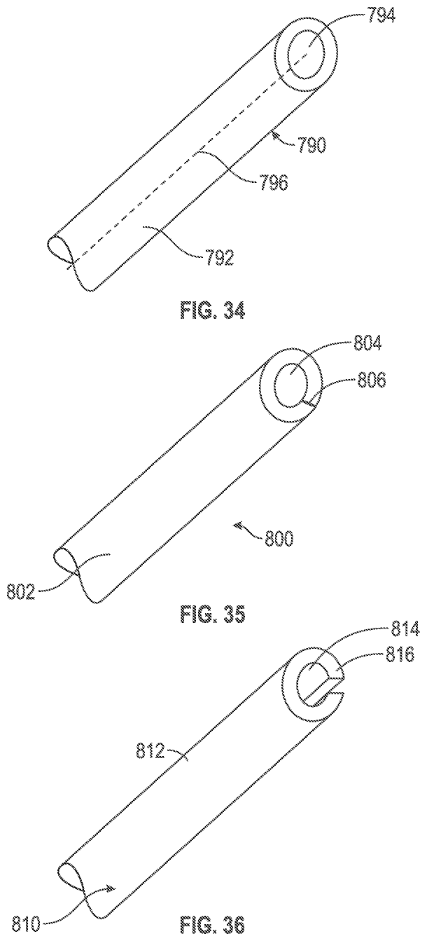

Elongate tubes 660A and 660B can include cut-away or peel-away features, as well as openable lumens described with reference to FIGS. 34 to 38B. Cut-away features can comprise axial sections of elongate tubes 660A and 660B that can be cut away by a surgeon or operator to trim elongate tubes 660A and 660B to desired lengths. Peel-away features can comprise portions of elongate tubes 660A and 660B that can be peeled away when no longer needed. For example, sleeves 764 and 774 of FIGS. 32A-33B can comprise portions of elongate tubes 660A and 660B that can be peeled away when it is desirable to deploy anchors 762 and 772.

Entry elements 662A and 662B can be connected to proximal ends of reentry devices 652A and 652B, respectively. Entry elements 662A and 662B can be configured to guide other components or instruments into elongate tubes 660A and 660B, respectively. In examples, entry elements 662A and 662B can comprise funnels or funnel-shaped bodies configured to gather distal ends of instruments and center said instruments with reentry devices 652A and 652B, respectively. In additional examples, entry elements 662A and 662B can be magnetic to pull instruments and devices into engagement with elongate tubes 660A and 660B. Entry elements 662A and 662B can be provided with magnets of opposite polarity to keep them apart from each other.

In additional examples, entry elements 662A and 662B and elongate tubes 660A and 660B can be threaded. As such, once an instrument or device is engaged with entry elements 662A and 662B and elongate tubes 660A and 660B, the instrument or device can be rotated to advance the instrument or device therethrough. Such features can be useful when it is difficult to advance the instrument or device solely through pushing due to complex geometries of the path through the anatomy and the like.

Reentry devices 652A and 652B are described as comprising tubes. However, other elongate bodies can be used. For example, elongate guide wires can be directly incorporated onto stent 650. Additionally, elongate rails can be attached to stent 650. Rails can comprise elongate bodies having cross-sections that allow mating channels to axially slide along the rails but prevent radial detachment of the channel from the rail. In examples, rails having t-shaped cross-sections can be used.

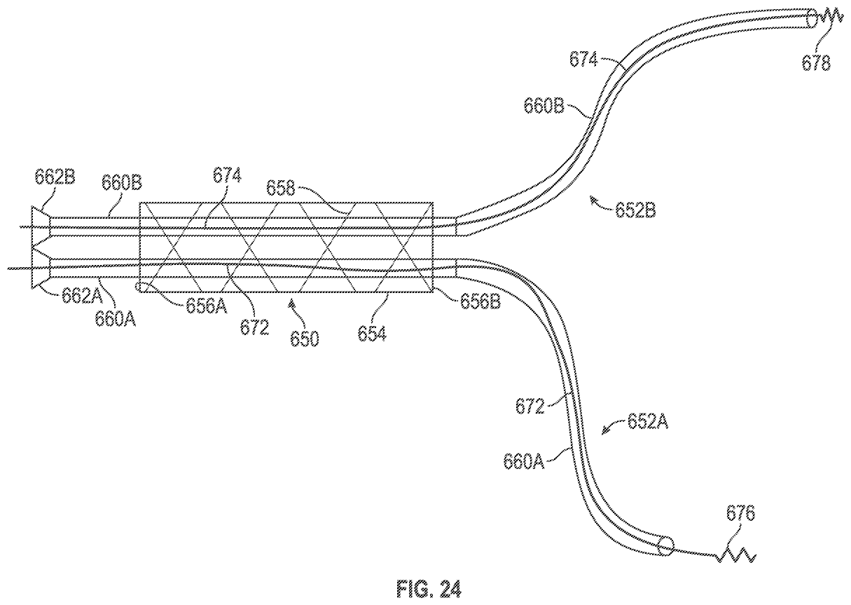

FIG. 24 is a schematic view of stent 650 of FIGS. 22 and 23 with reentry devices 652A and 652B in a deployed state. Reentry devices 652A and 652B can be extended from distal end of body 654 to position distal ends thereof further into anatomy.

Reentry device 652A can have guide wire 672 disposed therein and reentry device 652B can have guide wire 674 disposed therein. Guide wire 672 can have anchor 676 and guide wire 674 can have anchor 678. Anchors 676 and 678 can comprise devices configured to attach to tissue. In examples, anchors 676 and 678 can comprise screw devices that can be rotated by guide wires 672 and 674. However, other types of anchors can be used, such as those described with reference to FIGS. 32A-33B.

Guide wire 672 can be extended into entry device 662A to penetrate from the distal end of tube 660A. Guide wire 674 can be extended into entry device 662B to penetrate from the distal end of tube 660B. Guide wire 672 can be configured to position anchor 676 in anatomy. Guide wire 674 can be configured to position anchor 678 in the same or different anatomy. Anchors 676 and 678 can be configured to attach to tissue to fix guide wires 672 and 674 thereto. As discussed with reference to FIGS. 32A-33B, anchors 676 and 678 can be selectively deployed from tubes 660A and 660B. As such, other instruments such as scopes and forceps can be guided along guide wires 672 and 674 to the location of anchors 676 and 678.

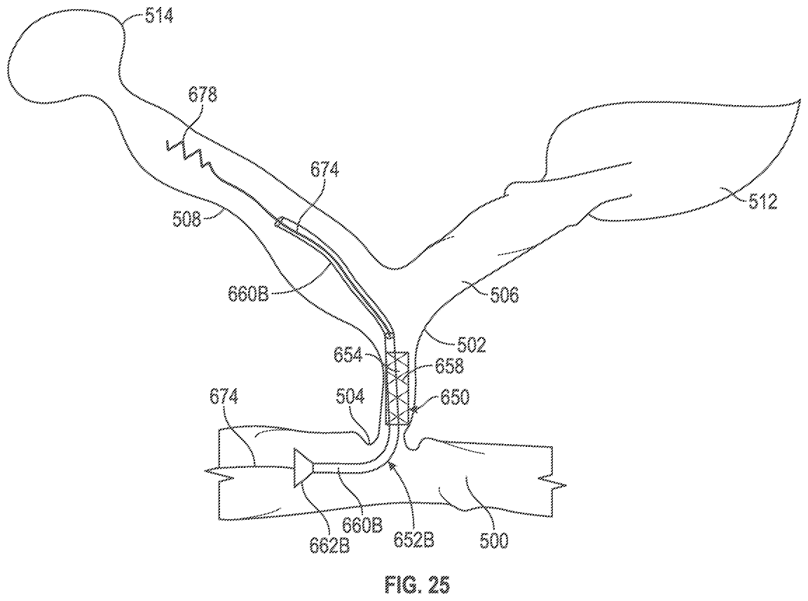

Tubes 660A and 660B can have different lengths to reach different anatomic locations. For example, tube 660B can be longer than tube 660A such that tube 660B can reach gallbladder 514 and tube 660A can reach pancreas 512. Additionally, tubes 660A and 660B can be curved or bent to extend through various anatomic ducts to reach a desired anatomy, as shown in FIG. 25. In examples, tube 660A and 660B can be used to receive fluid from an organ or can be used, via capillary action, to provide fluid, such as a medication, to an organ or anatomic area.

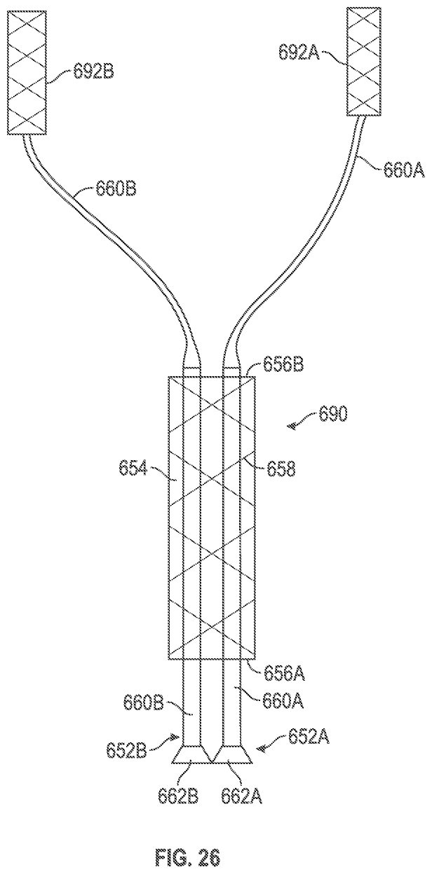

FIG. 25 is a schematic view of stent 650 of FIG. 24 with reentry device 652B deployed therefrom to reach gallbladder duct 508. Body 654 of stent 650 can be positioned in duodenal papilla 504 leading to common bile duct 502. Reentry device 652A is not shown for simplicity. Tube 660B can be bent or curved at approximately a ninety-degree angle to extend along duodenum 500 and then turn into common bile duct 502. As such, a device extending along duodenum 500 can be pushed into entry device 662B to be guided along by tube 660B. Tube 660B can thus induce a turning of the device inserted therein to guide that device into common bile duct 502. For example, guide wire 674 can be deployed from a scope, such as scope 14 (FIG. 4), into duodenum 500. Guide wire 674 can be advanced forward in the proximity of entry device 662B. Entry device 662B can be magnetic to pull guide wire 674 into a funnel shaped entryway. Once within entry device 662B, guide wire 674 can be pushed along tube 660B. The stiffness of tube 660B can be enough to cause guide wire 674 to turn and be pushed into common bile duct 502 and gallbladder duct 508 via tube 660B. When anchor 678 reaches the desired location near or within gallbladder 514, guide wire 674 can, for example, be rotated to cause anchor 678 to penetrate into tissue. As such, a treatment device, such as those described with reference to FIGS. 26-29B and others, can be slid along guide wire 674 to reach the location of anchor 674.

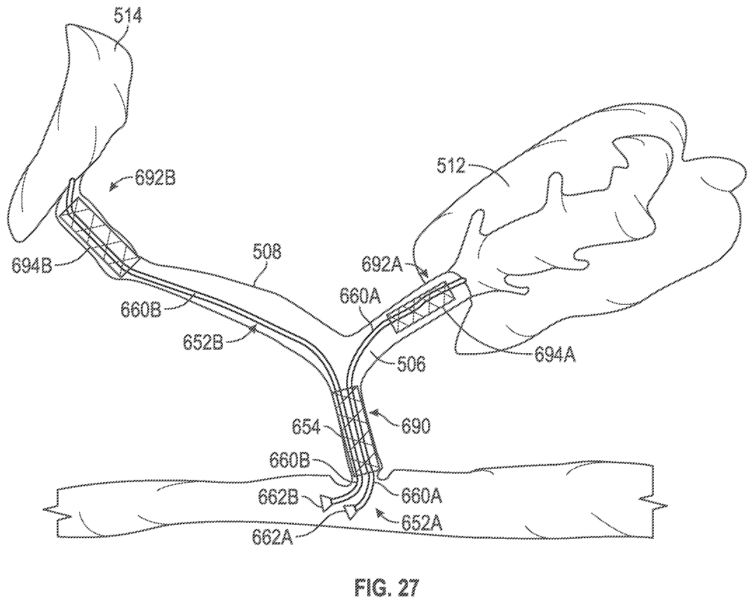

FIG. 26 is a schematic side view of stent 690 comprising distal stents 692A and 692B. Stent 690 can be configured similarly as stent 650 of FIG. 24 with the addition of distal stents 692A and 692B. Stent 692A can comprise body 694A and stent 692B can comprise body 694B. Bodies 694A and 694B can be attached to tubes 660A and 660B, respectively, as illustrated. In other examples, bodies 694A and 694B can be slit through tubes 660A and 660B for deployment distal of tubes 660A and 660B. Bodies 694A and 694B can comprise annular bodies configured to enlarge or hold open an anatomic duct. Thus, students 692A and 692B can allow larger stones than would otherwise to pass through anatomy. Bodies 694A and 694B can be configured as any of the stents described herein, including those described with reference to FIGS. 26-29B. In examples, bodies 694A and 694B can comprise annular bodies that can be collapsed during insertion and then expanded when positioned in a desired location to attach to an anatomic duct and expand the anatomic duct, including those described with reference to FIGS. 20A-21. In examples, bodies 694A and 694B can have smaller diameters than stent 690. In examples, bodies 694A and 694B can have different sizes for use in different anatomic locations, as shown in FIG. 27.

FIG. 27 is a schematic view of duodenum 500 having elongate reentry devices 652A and 652B attached to stent 690. Body 654 of stent 690 can be positioned in duodenal papilla 504 leading to common bile duct 502. Tube 660B can extend from stent 690 through gallbladder duct 508 to gallbladder 514. Tube 660A can extend from stent 690 through pancreas duct 506 to pancreas 512. Entry devices 662A and 662B can remain attached to tubes 660A and 660B, respectively, within duodenum 500 to facilitate placement of guidewires or other instruments or devices therethrough.

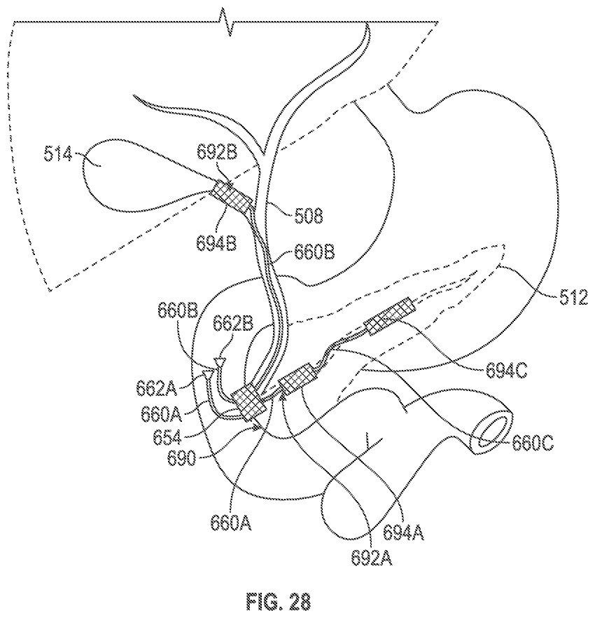

Stents 692A and 692B can be positioned within outlet orifices of gallbladder 514 and pancreas 512, respectively. Stents 692A and 692B can allow gallstones and pancreatic stones to pass out of their respective organs more freely. In additionally examples, stents 692A and 692B can be used to pump fluid from gallbladder 514 and pancreas 512. For example, as described with reference to FIG. 28, stents 692A and 692B can be configured to expand and contract to push fluid out of gallbladder 514 and pancreas 512. Additionally, stents 692A and 692B can be configured to generate head, such as via vibration, to allow for release or absorption of a medication. Furthermore, as described with reference to FIGS. 29A and 29B, stents 692A and 692B can be configured to treat or engage stones to allow the stones to be metabolized or passed out of the anatomy.

FIG. 28 is schematic view of duodenum 500 having stent 690 of FIG. 27 including therapeutic stent 694C connected to distal stent 694A via member 660C. Therapeutic stent 694C can be positioned inside of an organ, such as gall bladder 514 or pancreas 512. Therapeutic stent 694C can be provided with electrical leads, such as lead wires 534A and 534B described with reference to FIGS. 16A-16C and lead wires 552A and 552B described with reference to FIG. 17. The lead wires can allow therapeutic stent 694C to be actively controlled. In additional examples, therapeutic stent 694C can be provided without stent 694A being located between stent 694C and stent 690 (e.g., stent 694A can be omitted).

In examples, therapeutic stent 694C can be configured to expand and contract to provide pumping action. As such, therapeutic stent 694C can be configured to pump fluid into or out of an organ, or can provide pumping action to an organ to, for example, stimulate the organ to produce biological fluid. In examples, therapeutic stent 694C can be configured to expand and contract as described herein, such as by using magnetic activation.

In additional examples, therapeutic stent 694C can be configured to provide heating via lead wires. In examples, heating can be provided by resistance heating of lead wires and wires forming body stent 694C. In additional examples, heating can be provided by heating elements provided on stent 694C that are powered by electricity provided by the lead wires. In additional examples, heating can be provided by induction heating, such as with magnetic applicator 632 of FIG. 21. In examples, heating can be provided by vibration of stent 694C. Stent 694C can be vibrated by ultrasound.

Heating of therapeutic stent 694C can be used to provide fluid effects, such as by generating capillary action to draw fluid through stent 694C. Thus, stent 694C can be provided to push fluid out of gallbladder 514 or pancreas 512. Heating can also cause drugs delivered to stent 694C, such as through tube 660C, or provided on stent 694C as a coating to be evaporated into tissue. Additionally, as discussed above, heating of wire elements can be used to perform cutting, ablation and the like.

FIG. 29A is a schematic side view of a stent 700 of the present application configured to process biological matter, such as gallstones and pancreatic stones. FIG. 29B is an end view of stent 700 of FIG. 29A. FIGS. 29A and 29B are discussed concurrently.

In examples, stent 700 can comprise devices 702A and 702B and cutting edges 704A-704F in body 706. As can be seen in FIG. 29B, stent 700 can further comprise cutting elements 708A-708D extending across the interior of stent 700, and anchors 710A-710D.

Devices 702A and 702B can be configured to actively process biological matter passing through stent 700. In examples, stent 700 can be provided with electrical leads, such as lead wires 534A and 534B described with reference to FIGS. 16A-16C and lead wires 552A and 552B described with reference to FIG. 17. The lead wires can allow therapeutic stent 694C to be actively controlled. Devices 702A and 702B can be configured as rotary grinders to trim down or buff stones entering stent 700, such as via abrasion or abrading. In examples, the surface of the stones could be shaped to facilitate visualization with imaging, such as by making surfaces of the stone echogenic. Thus, stones larger than the inner diameter of stent 700 can be ground down to fit within stent 700 to allow passage out of gallbladder 514, for example.

In examples, devices 702A and 702B can have a coating or can emit a fluid that can dissolve gall stones.

In examples, devices 702A and 702B can be configured to emit laser beams that can dissolve or cut stones.

In examples, devices 702A and 702B can be configured to squeeze or compress stones to break the stones into smaller pieces.

In examples, cutting edges 704A-704F can provide trimming or shaving of stones passing through stent 700. Thus, the stones can become smaller while passing through stent 700 to allow for easier biological processing such as dissolving or passing out of the anatomy.

In examples, cutting elements 708A-708D can comprise wires that can be electrically activated to cause heating or vibration. Thus, stones entering stent 700 can be cut into smaller pieces to allow for easier biological processing. Vibration can be caused by excitation frequency. In examples, an additional instrument could be extended along guide wire to cause the vibration or activate device, etc. In additional examples, cutting elements 708A-708D can be used to filter or screen large sized stones from entering stent 700 to prevent blocking of stent 700. In such examples, cutting elements 708A-708D need not be configured to cut and do not need to be energized, but can simply comprise wires or the like to block free passage into stent 700. Such large stones can be removed by another procedure or with an additional instrument inserted into stent 700.

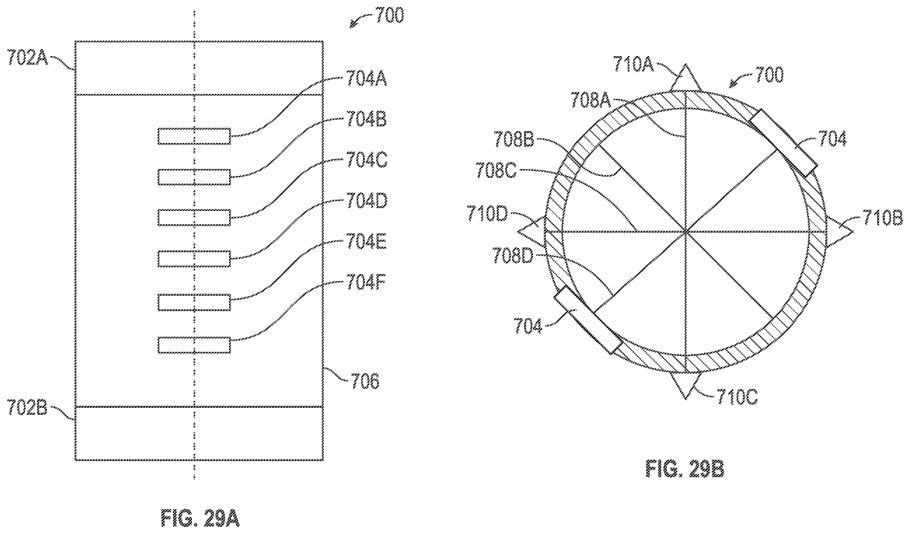

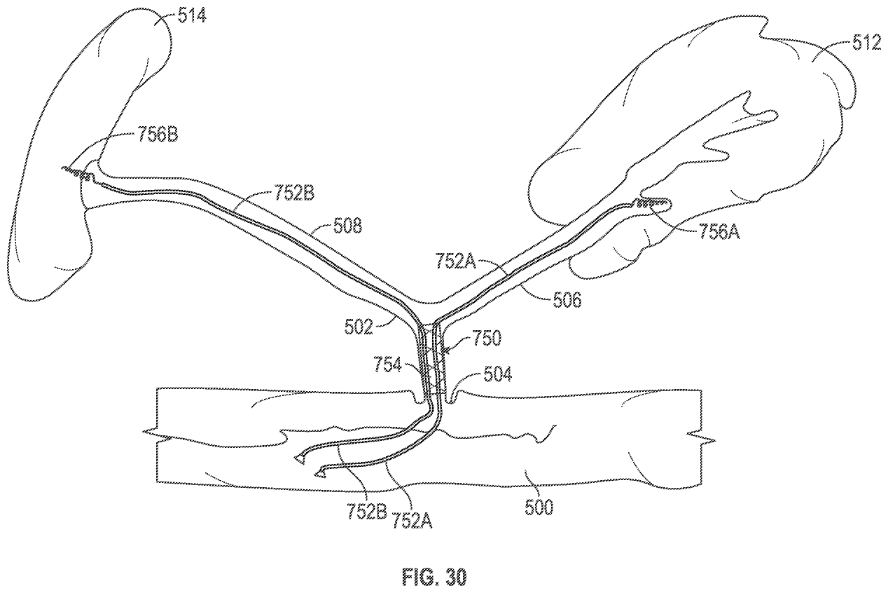

FIG. 30 is a schematic view of a duodenum 500 having an implantable device 750 of the present disclosure including monorails 752A and 752B. Implantable device 750 can comprise stent 754 through which monorails 752A and 752B can extend. Monorail devices can comprise wires or cables that can extend proximally of stent 754 into duodenum 500 and distally from stent 754 into pancreas duct 506 and gallbladder duct 508. The distal ends of monorails 752A and 752B can comprise anchors 756A and 756B, respectively.

Monorails 752A and 752B can be deployed using elongate tubes 660A and 660B as described with reference to FIGS. 22-25. Monorails 752A and 752B can comprise devices situated within anatomy to allow insertion of other devices without active steering and navigation. Monorails 752A and 752B can be left within the anatomy attached to stent 750 or can be withdrawn from stent 750 after a procedure is performed. Monorails 752A and 752B can thus be configured to be bioresorbable. In examples, stent 750 and monorails 752A and 752B can be inserted into anatomy using a suitable scope, such as by being guided through working channel 142 (FIG. 2) of scope 104.

FIG. 31 is a schematic view of a monorail 752A comprising corkscrew anchor 756A. Monorail 752A can comprise a wire or cable and anchor 756A can comprise a portion of the wire or cable wound into a coil or helix. Anchor 756A can be wound to have wider loops proximate monorail 752A that get progressively smaller toward the distal end of anchor 756A and finally terminating at the tip of monorail 752A. The tip of monorail 752A at the distal end of anchor 756A can be circumferentially oriented such that rotation of monorail 752A by an operator at a proximal end of monorail 752A can cause the tip of monorail 752A to penetrate tissue.

FIG. 32A is a schematic view of monorail 752A comprising deployable anchor 760 in a collapsed state. FIG. 32B is a schematic view of monorail 752A of FIG. 32A with deployable anchor 760 in an extended state. Anchor 760 can be deployed to enlarge in size and attach to tissue. FIGS. 32A and 32B are discussed concurrently.

Deployable anchor 760 can comprise anchor 762 and sleeve 764. Anchor 762 can comprise a plurality of deformable lobes 766A-766D. Each of deformable lobes 766A-766D can comprise a wire bent to have ends of the wire brought into proximity of each other to form a loop. Additionally, the distal portion of the loop can be curved outward from the ends of the wire forming the loop. The ends of the wire can be attached to monorail 752A such that each of the wires can be curved. When covered by sleeve 764, the curvature of the loop can be taken out such that the wire forming each of lobes 766A-766D can lie against monorail 752A as schematically illustrated in FIG. 32A. However, when sleeve 764 is retracted proximally along monorail 752A, the wire forming each of lobes 766A-766D can curve outward from monorail 752A, as schematically illustrated in FIG. 32B, to engage tissue.

Sleeve 764 can comprise tubing that surrounds anchor 762 to reduce the outer diameter size of deformable lobes 766A-766D. Sleeve 764 can extend from anchor 762 proximally along the length of monorail 752A. In examples, sleeve 764 can extend from anchor 762 all the way to an operator control at a proximal end. In examples, sleeve 764 can be just long enough to surround anchor 762 and provide the desired compaction of lobes 766A-766D and can be attached to an actuation wire that extends proximally to an operator control. Thus, either the proximal end of sleeve 764 or a control wire attached to sleeve 764 can be pulled proximally along the axis of monorail 752A to release lobes 766A-766D.

FIG. 33A is a schematic view of monorail 752A comprising deployable anchor 770 in a collapsed state. FIG. 33B is a schematic view of monorail 752A of FIG. 33A with deployable anchor 770 in an extended state. Anchor 770 can be deployed to enlarge in size and attach to tissue. FIGS. 33A and 33B are discussed concurrently.

Deployable anchor 770 can comprise anchor 772 and sleeve 774. Anchor 772 can comprise a plurality of extendable members 776A-776D. Each of extendable members 776A-776D can comprise a pointed protrusion extending from a base. The pointed protrusion can be biased radially outward from the base relative to the central axis of monorail 752A. When covered by sleeve 774, the pointed protrusions can be pushed closer to monorail 752A as schematically illustrated in FIG. 33A. However, when sleeve 774 is retracted proximally along monorail 752A, the pointed protrusions can extend radially outward from monorail 752A, as schematically illustrated in FIG. 33B, to engage tissue.

Sleeve 774 can comprise tubing that surrounds anchor 772 to reduce the outer diameter size of extendable members 776A-776D. Sleeve 774 can extend from anchor 772 proximally along the length of monorail 752A. In examples, sleeve 774 can extend from anchor 772 all the way to an operator control at a proximal end. In examples, sleeve 774 can be just long enough to surround anchor 772 and provide the desired compaction of extendable members 776A-776D and can be attached to an actuation wire that extends proximally to an operator control. Thus, either the proximal end of sleeve 774 or a control wire attached to sleeve 774 can be pulled proximally along the axis of monorail 752A to release members 776A-776D.

FIG. 34 is a schematic illustration of deployable member 790 comprising elongate shaft 792 comprising lumen 794 and perforations 796. Deployable member 790 can comprise tubes 660A and 660B of FIG. 22, sleeve 764 of FIGS. 32A and 32B, or sleeve 774 of FIGS. 33A and 33B. Elongate shaft 792 can comprise a flexible member fabricated of a polymer or another biocompatible material. Elongate shaft 792 can be made of resilient material that can be deformed or bent when subject to a load, but that will return to its original shape when the load is removed. Perforations 796 can extend along the length of elongate shaft 792 or just a select portion of shaft 792, such as a portion located proximate anchor 762 (FIGS. 32A and 32B) and anchor 772 (FIGS. 33A and 33B). Perforations 796 can comprise a series of small incisions extending through from the exterior of shaft 792 to lumen 794. Perforations 796 can allow shaft 792 to burst open along the line of perforation 796. As such, perforations 796 can allow passage of objects larger than the diameter of lumen 794 to pass through shaft 792. For example, the object can be pushed through lumen 794, bursting apart perforations 796 along the way.

FIG. 35 is a schematic illustration of deployable member 800 comprising elongate shaft 802 comprising lumen 804 and slit 806. Deployable member 800 can comprise tubes 660A and 660B of FIG. 22, sleeve 764 of FIGS. 32A and 32B, or sleeve 774 of FIGS. 33A and 33B. Elongate shaft 802 can comprise a flexible member fabricated of a polymer or another biocompatible material. Elongate shaft 802 can be made of resilient material that can be deformed or bent when subject to a load, but that will return to its original shape when the load is removed. Slit 806 can extend along the length of elongate shaft 802 or just a select portion of shaft 802, such as a portion located proximate anchor 762 (FIGS. 32A and 32B) and anchor 772 (FIGS. 33A and 33B). Slit 806 can comprise an incision extending through from the exterior of shaft 802 to lumen 804. Slit 806 can allow shaft 802 to spread open along the line of slit 806. As such, slit 806 can allow passage of objects larger than the diameter of lumen 804 to pass through shaft 802. For example, the object can be pushed through lumen 804, spreading apart split 806 along the way.

FIG. 36 is a schematic illustration of deployable member 810 comprising elongate shaft 812 comprising lumen 814 and gap 816. Deployable member 810 can comprise tubes 660A and 660B of FIG. 22, sleeve 764 of FIGS. 32A and 32B, or sleeve 774 of FIGS. 33A and 33B. Elongate shaft 812 can comprise a flexible member fabricated of a polymer or another biocompatible material. Elongate shaft 812 can be made of resilient material that can be deformed or bent when subject to a load, but that will return to its original shape when the load is removed. Gap 816 can extend along the length of elongate shaft 812 or just a select portion of shaft 812, such as a portion located proximate anchor 762 (FIGS. 32A and 32B) and anchor 772 (FIGS. 33A and 33B). Gap 816 can comprise an incision extending through from the exterior of shaft 812 to lumen 814. Gap 816 can allow shaft 812 to spread open along the line of gap 816. As such, gap 816 can allow passage of objects larger than the diameter of lumen 814 to pass through shaft 812. For example, the object can be pushed through lumen 814, spreading apart gap 816 along the way.

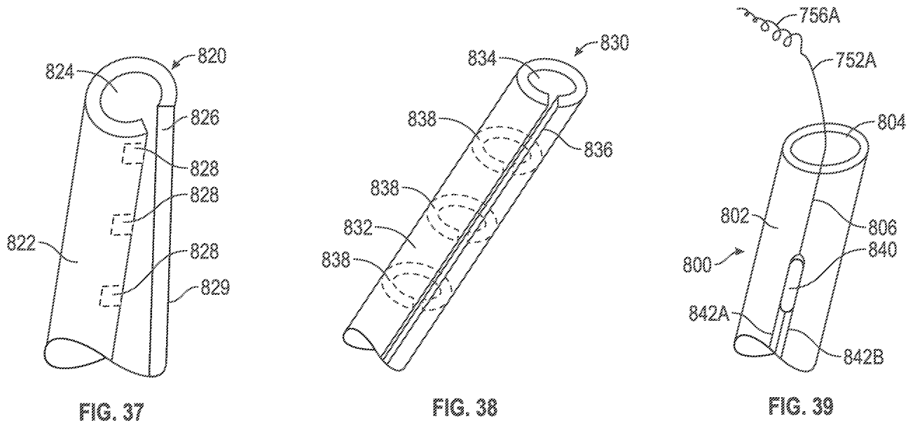

FIG. 37 is a schematic illustration of deployable member 820 comprising elongate shaft 822 comprising lumen 824 and gap 826. Gap 826 can include a plurality of magnetic members 828 and metallic strip 829. Magnetic members 828 can be attracted to metallic strip 829 via magnetic forces. Thus, at rest, magnetic members 828 can pull ends of elongate shaft 822 along gap 826 closed. However, when a device or object moves through elongate shaft 822 that is larger than lumen 824, magnetic members 828 can be pushed away from metallic strip 829 to allow the device or object to pass by. After the device or object passes by, magnetic members 828 can be pulled back into engagement with metallic strip 829 via magnetic attraction.

FIG. 38 is a schematic illustration of deployable member 830 comprising elongate shaft 832 comprising lumen 834 and gap 836. A plurality of c-shaped reinforcements 838 can be disposed on shaft 822 along lumen 824. C-shaped reinforcements 838 can be configured to have resiliency such that they can be deformed to widen the C-shape under a load from within lumen 834, but can return to their original shape when the load is removed. As such, devices or objects larger than lumen 834 can pass through shaft 832.

FIG. 39 is a schematic perspective view of an elongate deployable member 800 having treatment device 840 being slid therethrough to open elongate deployable member 800. Treatment device 840 can comprise any of the devices described herein. In examples, treatment device 840 can comprise a stent in a collapsed state. However, despite being in a collapsed state, treatment device 840 can be larger than lumen 804. As such, as treatment device 840 passes along slit 806, opposing faces 842A and 842B of shaft 802 can spread apart to allow treatment device 840 to pass therethrough.

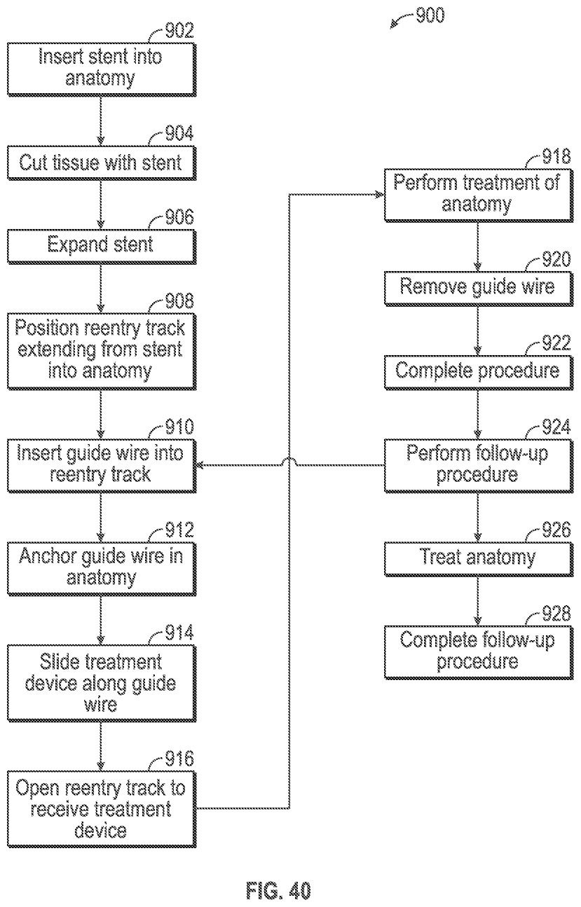

FIG. 40 is a block diagram illustrating methods 900 of implanting a reentry device of the present application having treatment devices.

At step 902, a stent can be inserted into anatomy. For example, stent 520 of FIG. 15 can be inserted into duodenal papilla 504 in duodenum 500. Stent 520 can be delivered to duodenum 500 using any suitable delivery device such as an endoscope. In examples, stent 520 can be delivered using direct peroral cholangioscopy system 100 of FIGS. 1A and 1B.

At step 904, tissue can be cut with the stent. For example, stent 550 of FIG. 17 can be used to electrically cut tissue or stent 600 of FIGS. 18 and 19 can be used to cut tissue. In examples, sphincter of Oddi 510 can be severed to allow duodenal papilla 504 to expand to receive the stent. Once inserted into place, the stent can be fixed to tissue, such as by using anchors 610A-610D of FIG. 19.

At step 906, the stent can be expanded. For example, device 630 of FIGS. 20A and 20B can be expanded within duodenal papilla 504 via magnetic action. In additional examples, stent 530 can be expanded via balloon 540.

At step 908, a reentry track or reentry device of the stent can be extended into anatomy. In examples, reentry devices 652A and 652B of FIG. 22 can be positioned within anatomic ducts adjoining the stent. Tubes 660A and 660B can have proximal ends positioned in duodenum 500 and distal ends positioned in gallbladder duct 508 and pancreas duct 506, respectively.

At step 910, a guide wire can be inserted into the reentry device. In examples, guide wire 674 (FIG. 24) can be positioned in tube 660B and guide wire 672 (FIG. 24) can be positioned in tube 660A.

At step 912, the guide wire can be anchored tO tissue. In examples, anchor 678 can be attached to gallbladder duct 508 or gallbladder 514 and anchor 676 can be attached to pancreas duct 506 or pancreas 512. Additionally, deployable or enlargeable anchors 760 and 770 of FIGS. 32A-33B can be used.

At step 914, a treatment device can be slid along the guide wire. For examples, stents 692A and 692 of FIG. 27 can be slid along guide wires 674 and 672. Also, stent 694C (FIG. 28) and stent 700 (FIGS. 29A and 29B) can be slit along guide wires to anatomy to be treated.

At step 916, a lumen of the reentry device can be opened to allow the treatment device to pass therethrough. For example, lumens 794, 804, 814824 and 834 of FIGS. 34-38 can be opened as the treatment device slides along a guide wire within a reentry tube. After the treatment devices passes by, the lumens can be closed using magnetic element 828 of FIG. 37, c-c-shaped reinforcements 838 of FIG. 38 or other means.

At step 918, anatomy can be treated using the treatment device. As discussed herein, the treatment device can provide pumping, heating, drug delivery, vibration, ablation, stone breaking and the like.

At step 920, the guide wire can be removed. For example, guide wires 674 and 672 can be withdrawn from reentry tubes 660A and 660B. In additional examples, guide wires 674 and 672 can be left within the anatomy for use in a subsequent procedure or to biologically resorb into the anatomy.

At step 922, the procedure can be completed. In particular, unused components can be removed from the anatomy and access portals and incisions in anatomy of the patient can be closed up to leave reentry components within the anatomy. Stent 520 (FIG. 15) and variations thereof described herein can be left in duodenal papilla 504. Additionally, reentry devices 652A and 652B can be left extended into desired anatomic ducts and turned into duodenum 500 to allow for later delivery of a treatment device and the like.

At step 924, a follow-up procedure can be performed. Method 900 can return to step 910 to use the previously implanted reentry device, such as by reopening the anatomy of the patient and inserting a scope to duodenum 500.

At step 926, anatomy can be treated during the follow-up procedure. Treatment performed at step 918 can be repeated or additional or different treatment can be performed, including the use of treatment devices described herein.

At step 928, the follow-up procedure can be completed. Components or devices that will no longer be used can be removed from the patient and components or devices that are intended to be subsequently used or used during a future procedure can be closed up inside the patient to facilitate later reentry.

### VARIOUS NOTES & EXAMPLES

Sheath for use with Guidewire for Reentry

Example 1 is a cholangioscopy system comprising: a guide sheath comprising a steerable lumen; and an endoscope comprising: an elongate shaft extending between a proximal end portion and a distal end face, the elongate shaft configured for displacement along the steerable lumen; a working tool lumen extending along the elongate shaft and exiting at the distal end face; an anchor lumen entering the elongate shaft between the proximal end portion and the distal end face and exiting the elongate shaft at the distal end face; and a non-axial lumen extending from the working tool lumen to an exterior of the elongate shaft at the distal end face.

In Example 2, the subject matter of Example 1 optionally includes an anchor wire extending through the anchor wire lumen.

In Example 3, the subject matter of Example 2 optionally includes wherein the anchor wire comprises: an elongate cable; and a tissue anchor located proximate a distal end of the anchor wire.

In Example 4, the subject matter of any one or more of Examples 1-3 optionally include a tissue retrieval device extendable from the working tool lumen.

In Example 5, the subject matter of Example 4 optionally includes the tissue retrieval device comprising a shaft capable of resiliently bending from extending along the working tool lumen to extending along the working tool lumen and into the non-axial lumen.

In Example 6, the subject matter of Example 5 optionally includes the tissue retrieval device comprising forceps.

In Example 7, the subject matter of any one or more of Examples 1-6 optionally include the non-axial lumen comprising a slit at the distal end face extending along the elongate shaft to intersect the working tool lumen.

In Example 8, the subject matter of Example 7 optionally includes the non-axial lumen extending through a side surface of the elongate shaft.

In Example 9, the subject matter of any one or more of Examples 1-8 optionally include the non-axial lumen extending within the distal-most ten percent of the length of the elongate shaft.

In Example 10, the subject matter of any one or more of Examples 1-9 optionally include the endoscope including steering capabilities.

Example 11 is a method of performing a direct peroral cholangioscopy procedure, the method comprising: inserting an endoscope into a guide sheath; inserting the guide sheath and endoscope into the duodenum of a patient; extending a tissue retrieval device through the endoscope into the common bile duct of the patient; extending an anchor wire into the common bile duct; collecting biological matter with the tissue retrieval device; and withdrawing the cholangioscope and tissue retrieval device from the common bile duct along the anchor wire.

In Example 12, the subject matter of Example 11 optionally includes inserting the guide sheath and endoscope into the duodenum of a patient comprises using the guide sheath to steer the cholangioscope.

In Example 13, the subject matter of Example 12 optionally includes steering the cholangioscope using native steering capabilities of the cholangioscope.

In Example 14, the subject matter of Example 13 optionally includes steering the cholangioscope by pushing off of the guide sheath.

In Example 15, the subject matter of any one or more of Examples 11-14 optionally include extending the anchor wire into the common bile duct by attaching the anchor wire to tissue of the common bile duct.

In Example 16, the subject matter of any one or more of Examples 11-15 optionally include reinserting the cholangioscope into the common bile duct by sliding the cholangioscope along the anchor wire.

In Example 17, the subject matter of Example 16 optionally includes removing tissue from the tissue retrieval device before reinserting the cholangioscope.

In Example 18, the subject matter of Example 17 optionally includes positioning a distal portion of the tissue retrieval device within a non-axial lumen of the cholangioscope after withdrawing the cholangioscope and tissue retrieval device from the common bile duct along the anchor wire.

In Example 19, the subject matter of Example 18 optionally includes positioning a distal portion of the tissue retrieval device within a non-axial lumen of the cholangioscope by bending a portion of a shaft of the tissue retrieval device extending from a working tool lumen to extend through the non-axial lumen.

In Example 20, the subject matter of Example 19 optionally includes the non-axial lumen comprises a slot in a distal end of the cholagioscope connecting the working tool lumen to a side surface of the cholangioscope.

Stent-Type Device for Reentry

Example 1 is a system for providing repeatable entry into an anatomic area of a patient, the system comprising: a stent comprising an annular body; and a reentry track extending through the stent, the reentry track comprising: an elongate body comprising proximal and distal ends extending out of the annular body.

In Example 2, the subject matter of Example 1 optionally includes the elongate body comprising a tubular body defining a lumen.

In Example 3, the subject matter of Example 2 optionally includes the tubular body being openable.

In Example 4, the subject matter of Example 3 optionally includes the tubular body comprising a slit extending along a length of the tubular body.

In Example 5, the subject matter of Example 4 optionally includes the slit being closeable.

In Example 6, the subject matter of Example 5 optionally includes wherein the tubular body comprises magnets positioned along the slit.

In Example 7, the subject matter of any one or more of Examples 5-6 optionally includes the tubular body comprises springs configured to bias the slit to a closed position.

In Example 8, the subject matter of any one or more of Examples 3-7 optionally includes the tubular body comprising a perforation extending along a length of the tubular body.

In Example 9, the subject matter of any one or more of Examples 2-8 optionally includes the tubular body comprising threading extending along the lumen.

In Example 10, the subject matter of any one or more of Examples 2-9 optionally includes an entry device attached to a proximal end of the tubular body.

In Example 11, the subject matter of Example 10 optionally includes the entry device comprising a funnel.

In Example 12, the subject matter of Example 11 optionally includes the funnel including internal threading.

In Example 13, the subject matter of any one or more of Examples 11-12 optionally includes the funnel being magnetic.

In Example 14, the subject matter of any one or more of Examples 2-13 optionally includes a guide wire extending into the lumen of the tubular body; and an anchor attached to a distal end portion of the guide wire.

In Example 15, the subject matter of Example 14 optionally includes the anchor comprising a screw.

In Example 16, the subject matter of any one or more of Examples 14-15 optionally includes the anchor comprising an enlargeable body, wherein the anchor can be retracted into the lumen of the tubular body to contract the enlargeable body and extended from the lumen to allow the enlargeable body to expand.

In Example 17, the subject matter of Example 16 optionally includes the enlargeable body comprising a plurality of bendable lobes.

In Example 18, the subject matter of any one or more of Examples 16-17 optionally includes the enlargeable body comprising a plurality of extendable projections.

In Example 19, the subject matter of any one or more of Examples 1-18 optionally includes an intervention device configured to move along the reentry track.

In Example 20, the subject matter of Example 19 optionally includes the intervention device comprising a treatment stent.

In Example 21, the subject matter of Example 20 optionally includes the treatment stent being configured to provide pumping action in the anatomic area.

In Example 22, the subject matter of Example 21 optionally includes the treatment stent being configured to expand and contract.

In Example 23, the subject matter of any one or more of Examples 20-22 optionally includes the treatment stent device comprising a stone processor.

In Example 24, the subject matter of Example 23 optionally includes the stone processor being configured to chemically treat stones.

In Example 25, the subject matter of any one or more of Examples 23-24 optionally includes the stone processor being configured to mechanically reduce the size of stones.

In Example 26, the subject matter of any one or more of Examples 20-25 optionally includes the treatment stent being configured to provide heating.

In Example 27, the subject matter of any one or more of Examples 20-26 optionally includes a balloon positioned within the treatment stent.

In Example 28, the subject matter of any one or more of Examples 1-27 optionally includes the stent being configured to cut tissue.

In Example 29, the subject matter of Example 28 optionally includes the stent comprising a mechanical cutting blade.

In Example 30, the subject matter of any one or more of Examples 28-29 optionally includes the stent comprising an electric cutting blade.

In Example 31, the subject matter of Example 30 optionally includes the electric cutting blade comprising a pair of electric lead wires that are configured to be pulled out of the stent after use.

In Example 32, the subject matter of any one or more of Examples 1-31 optionally includes the stent being selectively expandable.

In Example 33, the subject matter of Example 32 optionally includes a plurality of magnets located on the stent; and a magnetic applicator configured to move along the reentry track to magnetically expand the stent.

Example 34 is a method for implanting a treatment device in anatomy, the method comprising: implanting a stent in an anatomic opening; positioning a reentry track extending from the stent into an anatomic passageway; sliding the treatment device along the reentry track to position the treatment device in the anatomic passageway; and treating the anatomy with the treatment device.

In Example 35, the subject matter of Example 34 optionally includes implanting the stent in the anatomic opening by cutting tissue with the stent.

In Example 36, the subject matter of Example 35 optionally includes cutting tissue with the stent by electrically cutting tissue with bi-polar lead wires.

In Example 37, the subject matter of Example 36 optionally includes removing the bi-polar lead wires from the stent.

In Example 38, the subject matter of any one or more of Examples 34-37 optionally includes implanting the stent in an anatomic opening by expanding the stent.

In Example 39, the subject matter of Example 38 optionally includes expanding the stent by magnetically expanding the stent.

In Example 40, the subject matter of any one or more of Examples 34-39 optionally includes positioning the reentry track extending from the stent into the anatomic passageway by positioning a tube in the anatomic passageway.

In Example 41, the subject matter of Example 40 optionally includes inserting a guide wire through the reentry track and into the anatomic passageway.

In Example 42, the subject matter of Example 41 optionally includes inserting the guide wire through the reentry track and into the anatomic passageway by attaching an anchor to the anatomic passageway.

In Example 43, the subject matter of Example 42 optionally includes attaching an anchor to the anatomic passageway by expanding the anchor to attach to tissue.

In Example 44, the subject matter of Example 43 optionally includes expanding the anchor to attach to tissue by pushing the expandable anchor out of the tube to allow the expandable anchor to expand.

In Example 45, the subject matter of any one or more of Examples 41-44 optionally includes sliding the treatment device along the reentry track to position the treatment device in the anatomic passageway by sliding a treatment stent along the guide wire.

In Example 46, the subject matter of Example 45 optionally includes sliding the treatment stent along the guide wire by opening the tube.

In Example 47, the subject matter of Example 46 optionally includes opening the tube by spreading a gap in the tube.

In Example 48, the subject matter of any one or more of Examples 46-47 optionally include opening the tube by bursting a perforation extending along the tube.

In Example 49, the subject matter of any one or more of Examples 46-48 optionally includes reclosing the tube via magnetic closure elements.

In Example 50, the subject matter of any one or more of Examples 46-49 optionally includes reclosing the tube via resilient closure elements.

In Example 51, the subject matter of any one or more of Examples 34-50 optionally include treating the anatomy with the treatment device by pumping anatomy in which the treatment device is deployed.

In Example 52, the subject matter of Example 51 optionally includes wherein pumping anatomy in which the treatment device is deployed by expanding and contracting a stent.

In Example 53, the subject matter of any one or more of Examples 34-52 optionally includes treating the anatomy with the treatment device by treating a stone within the anatomy to facilitate passing through the treatment device.

In Example 54, the subject matter of Example 53 optionally includes treating the stone within the anatomy to facilitate passing through the treatment device by chemically dissolving the stones.

In Example 55, the subject matter of any one or more of Examples 53-54 optionally includes treating the stone within the anatomy to facilitate passing through the treatment device by mechanically reducing size of the stones.

In Example 56, the subject matter of any one or more of Examples 34-55 optionally includes treating the anatomy with the treatment device by heating the treatment device.

In Example 57, the subject matter of Example 56 optionally includes heating the treatment device by vibrating the treatment device.

In Example 58, the subject matter of any one or more of Examples 34-57 optionally includes inflating a balloon within the stent to expand the stent.

In Example 59, the subject matter of any one or more of Examples 34-58 optionally includes sliding the treatment device along the reentry track by funneling the treatment device into the reentry track.

In Example 60, the subject matter of any one or more of Examples 34-59 optionally include screwing the treatment device into the reentry track.

In Example 61, the subject matter of any one or more of Examples 34-60 optionally includes magnetically attracting the treatment device to the reentry track.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A cholangioscopy system comprising:
a guide sheath comprising a steerable lumen; and
an endoscope comprising:
an elongate shaft extending between a proximal end portion and a distal end face, the elongate shaft configured for displacement along the steerable lumen;
a working tool lumen extending along the elongate shaft and exiting at the distal end face;
an anchor lumen entering the elongate shaft between the proximal end portion and the distal end face and exiting the elongate shaft at the distal end face; and
a non-axial lumen extending from the working tool lumen to an exterior of the elongate shaft at the distal end face.

2. The cholangioscopy system of claim 1, further comprising an anchor wire extending through the anchor wire lumen.

3. The cholangioscopy system of claim 2, wherein the anchor wire comprises:
an elongate cable; and
a tissue anchor located proximate a distal end of the anchor wire.

4. The cholangioscopy system of claim 1, further comprising a tissue retrieval device extendable from the working tool lumen.

5. The cholangioscopy system of claim 4, wherein the tissue retrieval device comprises a shaft capable of resiliently bending from extending along the working tool lumen to extending along the working tool lumen and into the non-axial lumen.

6. The cholangioscopy system of claim 5, wherein the tissue retrieval device comprises forceps.

7. The cholangioscopy system of claim 1, wherein the non-axial lumen comprises a slit at the distal end face extending along the elongate shaft to intersect the working tool lumen.

8. The cholangioscopy system of claim 7, wherein the non-axial lumen extends through a side surface of the elongate shaft.

9. The cholangioscopy system of claim 1, wherein the non-axial lumen extends within the distal-most ten percent of the length of the elongate shaft.

10. The cholangioscopy system of claim 1, wherein the endoscope includes steering capabilities.

11. A method of performing a direct peroral cholangioscopy procedure, the method comprising:
inserting an endoscope into a guide sheath;
inserting the guide sheath and endoscope into the duodenum of a patient;
extending a tissue retrieval device through the endoscope into the common bile duct of the patient;
extending an anchor wire into the common bile duct;
collecting biological matter with the tissue retrieval device; and
withdrawing the cholangioscope and tissue retrieval device from the common bile duct along the anchor wire.

12. The method of claim 11, wherein inserting the guide sheath and endoscope into the duodenum of a patient comprises using the guide sheath to steer the cholangioscope.

13. The method of claim 12, further comprising steering the cholangioscope using native steering capabilities of the cholangioscope.

14. The method of claim 13, wherein steering the cholangioscope comprises pushing off of the guide sheath.

15. The method of claim 11, wherein extending the anchor wire into the common bile duct comprises attaching the anchor wire to tissue of the common bile duct.

16. The method of claim 11, further comprising reinserting the cholangioscope into the common bile duct by sliding the cholangioscope along the anchor wire.

17. The method of claim 16, further comprising removing tissue from the tissue retrieval device before reinserting the cholangioscope.

18. The method of claim 17, further comprising positioning a distal portion of the tissue retrieval device within a non-axial lumen of the cholangioscope after withdrawing the cholangioscope and tissue retrieval device from the common bile duct along the anchor wire.

19. The method of claim 18, wherein positioning a distal portion of the tissue retrieval device within a non-axial lumen of the cholangioscope comprises bending a portion of a shaft of the tissue retrieval device extending from a working tool lumen to extend through the non-axial lumen.

20. The method of claim 19, wherein the non-axial lumen comprises a slot in a distal end of the cholagioscope connecting the working tool lumen to a side surface of the cholangioscope.
